(19) United States
(12) Patent Application Publication
(10) Pub. No.: US 2013/0108665 A1
(43) Pub. Date: May 2, 2013

(54) RECOMBINANT TUMOR VACCINE AND METHOD OF PRODUCING SUCH VACCINE

(71) Applicant: Min Liang, Shanghai (CN)

(72) Inventor: Min Liang, Shanghai (CN)

(73) Assignee: TOT SHANGHAI R&D CENTER CO., LTD., Shanghai (CN)

(21) Appl. No.: 13/805,237

(22) PCT Filed: Jun. 30, 2011
(86) PCT No.: PCT/CN2011/076668

§ 371 Date: Jan. 15, 2013

(30) Foreign Application Priority Data
Jun. 30, 2010 (CN) PCT/CN2010/074823

(51) Int. Cl. A61K 31/7088

(52) U.S. Cl. CPC A61K 31/7088
USPC 424/277.1; 435/320.1; 435/254.2; 435/325

(57) ABSTRACT

The present disclosure provides tumor vaccines useful for preventing and treating tumors and cancers. The tumor vaccines may contain nucleic acids encoding for antigen presenting peptides, cytokines and other factors useful for preventing and treating tumors and cancers, or expression vectors or viruses containing such nucleic acids, or host cells containing such nucleic acids or expression vectors.

### BACKGROUND

Antigen presentation is a process in adaptive immune responses, during which antigen-presenting cells capture antigens and present the antigens on their surface for recognition by T cells. Antigens are displayed on the surface of antigen-presenting cells together with major histocompatibility complex (MHC) molecules, which can be recognized and bound by T cell receptors and thus promote antigen presentation. Once the T cells recognize the antigens presented on the cell surface, they are activated and generate immune responses against the antigens. Through antigen presentation, T cells can generate immune responses towards exogenous antigens as well as endogenous antigens.

Cytokines are important players in the immune response regulation. Cytokines are small proteins secreted by cells in the immune system that can function to transmit signals for regulating the immune system. Different cytokines can carry different signals. For example, some cytokines can stimulate proliferation or maturation of immune cells; some cytokines can direct the migration of immune cells; and some cytokines can enhance cell-based immune responses.

Cancers or tumors are believed to be associated with, at least partially, weakened or deficient immune responses to cancerous changes in people. Various therapies have been used to treat cancers including gene therapy. Gene therapy for cancer treatment may involve delivery of genes that can kill or inhibit cancer cells, genes that can enhance immune responses against cancer cells, genes that can repair or replace the mutated or altered genes, or genes that can make cancer cells more sensitive to chemotherapy or radiation therapy.

### SUMMARY

The present disclosure relates to recombinant tumor vaccines useful for preventing and treating tumors and cancers. The tumor vaccines may contain nucleic acids encoding for antigen presenting peptides, cytokines and other factors useful for preventing and treating tumors and cancers, and/or expression vectors containing such nucleic acids, and/or host cells containing such nucleic acids and/or expression vectors. The present disclosure also relates to pharmaceutical compositions containing the tumor vaccines and methods of preventing and treating tumors and cancers using the tumor vaccines.

In one aspect, the present disclosure provides a recombinant tumor vaccine including a composition of a first polynucleotide encoding an antigen presenting polypeptide, a second polynucleotide encoding a cytokine, and at least one promoter, where the at least one promoter is operably linked to at least one of the first polynucleotide and the second polynucleotide.

In another aspect, the present disclosure provides a recombinant tumor vaccine including an expression vector having a first polynucleotide segment encoding an antigen presenting polypeptide, a second polynucleotide segment encoding a cytokine, and at least one promoter, where the at least on promoter is operably linked to at least one of the first polynucleotide segment and the second polynucleotide segment.

In another aspect, the present disclosure provides a pharmaceutical composition containing a pharmaceutically acceptable carrier and a tumor vaccine containing a first polynucleotide encoding an antigen presenting polypeptide, a second polynucleotide encoding a cytokine, and at least one promoter, where the at least one promoter is operably linked to at least one of the first polynucleotide and the second polynucleotide.

In another aspect, the present disclosure provides a method of treating a tumor, including administering into a subject a pharmaceutical composition containing a pharmaceutically acceptable carrier and a tumor vaccine containing a first polynucleotide encoding an antigen presenting polypeptide, a second polynucleotide encoding a cytokine, and at least one promoter, where the at least one promoter is operably linked to at least one of the first polynucleotide and the second polynucleotide.

In another aspect, the present disclosure provides a pharmaceutical composition containing a pharmaceutically acceptable carrier and a tumor vaccine containing a first polynucleotide segment encoding an antigen presenting polypeptide, a second polynucleotide segment encoding a cytokine, and at least one promoter, where the at least on promoter is operably linked to at least one of the first polynucleotide segment and the second polynucleotide segment.

In another aspect, the present disclosure provides a method of treating a tumor, including administering into a subject a pharmaceutical composition containing a pharmaceutically acceptable carrier and a tumor vaccine containing a first polynucleotide segment encoding an antigen presenting polypeptide, a second polynucleotide segment encoding a cytokine, and at least one promoter, where the at least on promoter is operably linked to at least one of the first polynucleotide segment and the second polynucleotide segment.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 shows a schematic map of the expression vector pAd-HSP-hGM-CSF.

FIG. 2 shows a schematic map of the genome of the adenovirus Ad-HSP-hGM-CSF.

FIG. 3 shows a schematic map of the genome of the adenovirus Ad-HSP-mGM-CSF.

FIG. 4 shows the mGM-CSF expression levels determined by ELISA in B 16 cells (a), Hep3B cells (b), and Hela cells (c), which are infected with Ad-HSP-mGM-CSF under the Multiplicity of Infection (MOI) of 10, 100 and 1000, respectively.

FIG. 5 shows the hHSP70 expression levels determined by ELISA in B 16 cells (a), Hep3B cells (b), and Hela cells (c), which are infected with Ad-HSP-mGM-CSF under the MOIs of 10, 100 and 1000, respectively.

FIG. 6 shows the cytotoxic effects of Ad-HSP-hGM-CSF on different tumor cells at a multiplicity of infection at 300.

FIG. 7 shows the cytotoxic effects of Ad-HSP-hGM-CSF on different tumor cells at a multiplicity of infection at 100.

FIG. 8 shows the cytotoxic effects of Ad-HSP-hGM-CSF on different tumor cells at a multiplicity of infection at 30.

FIG. 9 shows the cytotoxicity of Ad-HSP-mGM-CSF on 7 different types of cells.

FIG. 10 shows the changes of tumor sizes in mice treated with Ad-HSP-mGM-CSF.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The Nucleic Acid Composition

The present disclosure relates to a recombinant tumor vaccine having a composition containing a first polynucleotide encoding an antigen presenting polypeptide, a second polynucleotide encoding a cytokine, and at least one promoter, where the at least one promoter is operably linked to at least one of the first polynucleotide and the second polynucleotide.

The term “recombinant” as used herein refers to artificial manipulation of one or more biological molecules such as polynucleotide or polypeptide molecules using one or more molecular biology techniques to make such biological molecule(s) into something other than its natural state.

The term “polynucleotide” or “nucleic acid” as used herein refers to ribonucleic acids (RNA), deoxyribonucleic acids (DNA), or mixed ribonucleic acids-deoxyribonucleic acids such as DNA-RNA hybrids. The polynucleotide or nucleic acid may be single stranded or double stranded DNA or RNA or DNA-RNA hybrids. The polynucleotide or nucleic acid may be linear or circular.

The term “encoding” or “encoding for” as used herein refers to being capable of being transcribed into mRNA and/or translated into a peptide or protein. An “encoding sequence” or a “gene” refers to a polynucleotide sequence that encodes for an mRNA, a peptide or a protein. These two terms are used interchangeably in the present disclosure.

The term “mRNA” as used herein refers to a RNA molecule that is complementary to a template DNA sequence, and can serve as the template for protein synthesis.

The term “antigen presenting polypeptide” as used herein refers to a polypeptide or a protein that can bind to an antigen, present the antigen to the antigen presenting cell and facilitate the recognition of the antigen by an antigen presenting cell.

The term “antigen presenting cell” as used herein refers to a cell that is capable of displaying an antigen with major histocompatibility complex (MHC) on its surface, in such a way that T cells can recognize the displayed antigen. The displayed antigen can be recognized by immune cells such as T cells which consequently generate an immune response against the antigen. In certain embodiments, the antigen presenting cells are dendritic cells, macrophages, B-cells or epithelia cells. In an illustrative embodiment, the antigen presenting cells are dendritic cells.

The term “operably linked” as used herein refers to that the encoding sequence is directly or indirectly linked to or associated with one or more regulatory sequences in a manner that allows expression of the encoding sequence.

The term “expression” as used herein refers to transcription of an encoding sequence into mRNA and/or translation of an encoding sequence into a peptide or protein.

The term “transcription” as used herein refers to the process of RNA synthesis where a DNA sequence is read by a RNA polymerase to produce a RNA chain complementary to the DNA sequence.

The term “translation” as used herein refers to the process of peptide or protein synthesis where an mRNA sequence is read by a ribosome to produce an amino acid chain according to the amino acid codons contained in the mRNA sequence.

The term “regulatory sequence” as used herein refers to any nucleotide sequence that is necessary or advantageous for the expression of an encoding sequence. A regulatory sequence may include, but is not limited to, one or more promoters, enhancers, transcription terminators, polyadenylation sequences, and internal ribosome entry sites.

The term “promoter” as used herein refers to a polynucleotide sequence that can control transcription of an encoding sequence. The promoter sequence includes specific sequences that are sufficient for RNA polymerase recognition, binding and transcription initiation. In addition, the promoter sequence may include sequences that modulate this recognition, binding and transcription initiation activity of RNA polymerases. The promoter may affect the transcription of a gene located on the same nucleic acid molecule as itself or a gene located on a different nucleic acid molecule as itself. Functions of the promoter sequences, depending upon the nature of the regulation, may be constitutive or inducible by a stimulus. A “constitutive” promoter refers to a promoter that functions to continually activate gene expression in host cells. An “inducible” promoter refers to a promoter that activates gene expression in host cells in the presence of certain stimulus or stimuli.

The term “enhancer” as used herein refers to a nucleotide sequence that increases the transcription and/or translation of the encoding sequence. The enhancer may be operably linked to the 5′ terminus or 3′ terminus of an encoding sequence. Any enhancer that is functional in eukaryotic cells may be used in the present disclosure. Illustrative examples of enhancers include, without limitation, the simian virus 40 (SV40) early gene enhancer, the enhancer derived from the long terminal repeat (LTR) of Rous Sarcoma virus, and the enhancer derived from human cytomegalovirus (CMV).

The term “transcription terminator” as used herein refers to a nucleotide sequence recognized by a eukaryotic cell RNA polymerase to terminate transcription. The terminator sequence may be operably linked to the 3′ terminus of an encoding sequence. In certain embodiments, the terminator may comprise a signal for the cleavage of the RNA such that a polyadenylation site on the RNA can be exposed. Any terminator sequence that is functional in eukaryotic cells may be used in the present disclosure. Illustrative examples of terminator sequences include, without limitation, termination sequences derived from a virus such as the SV40 terminator, and termination sequences derived from known genes such as the bovine growth hormone terminator sequence.

The term “polyadenylation sequence” as used herein refers to a nucleotide sequence which, when transcribed, is recognized by eukaryotic cells as a signal to add polyadenosine residues to the transcribed mRNA. The polyadenylation sequence may be operably linked to the 3′ terminus of an encoding sequence. Any polyadenylation sequence which is functional in eukaryotic cells may be used in the present disclosure. Illustrative examples of polyadenylation sequences include, without limitation, AAUAAA, and SV40 polyadenylation signal.

The term “internal ribosome entry site” (IRES), as used herein refers to a nucleotide sequence which allows translation initiation from the middle of an mRNA sequence. IRES can be used to separate two or more encoding sequences on one polynucleotide molecule so as to allow separate translation of the two or more encoded products. IRES may be operably linked at a position after the 3′ terminus of a first encoding sequence and before the 5′ terminus of a second encoding sequence. Any IRES sequence which is functional in eukaryotic cells may be used in the present disclosure. Illustrative examples of IRES may include, without limitation, picornavirus IRES, pestivirus IRES, aphthovirus IRES, hepatitis A IRES, and hepatitis C IRES.

The present disclosure provides a composition containing a first polynucleotide encoding an antigen presenting polypeptide and a second polynucleotide encoding a cytokine. In certain embodiments, the first polynucleotide and the second polynucleotide are on different nucleic acid molecules. In certain embodiments, the first polynucleotide and the second polynucleotide are on the same nucleic acid molecules. In certain embodiments, a single promoter controls the expression of the first polynucleotide and the second polynucleotide. In certain embodiments, different promoters control the expression of the first polynucleotide and the second polynucleotide.

In certain embodiments, the first polynucleotide and the second polynucleotide are operably linked to one promoter, which controls the expression of the first and second polynucleotide, and in which the first polynucleotide segment and the second polynucleotide segment are separated by an IRES sequence.

In certain embodiments, the first polynucleotide is operably linked to a first promoter that controls the expression of the first polynucleotide, and the second polynucleotide is operably linked to a second promoter that controls the expression of the second polynucleotide. The first promoter and the second promoter can be the same or different.

In certain embodiments, the promoters of the present disclosure are eukaryotic promoters such as the promoters from CMV (e.g. the CMV immediate early promoter (CMV promoter)), epstein barr virus (EBV) promoter, human immunodeficiency virus (HIV) promoter (e.g. the HIV long terminal repeat (LTR) promoter), moloney virus promoter, mouse mammary tumor virus (MMTV) promoter, rous sarcoma virus (RSV) promoter, SV40 early promoter, promoters from human genes such as human myosin promoter, human hemoglobin promoter, human muscle creatine promoter, human metalothionein beta-actin promoter, human ubiquitin C promoter (UBC), mouse phosphoglycerate kinase 1 promoter (PGK), human thymidine kinase promoter (TK), human elongation factor 1 alpha promoter (EF1A), cauliflower mosaic virus (CaMV) 35S promoter, E2F-1 promoter (promoter of E2F1 transcription factor 1), promoter of alpha-fetoprotein, promoter of cholecystokinin, promoter of carcinoembryonic antigen, promoter of C-erbB2/neu oncogene, promoter of cyclooxygenase, promoter of CXC-Chemokine receptor 4 (CXCR4), promoter of human epididymis protein 4 (HE4), promoter of hexokinase type II, promoter of L-plastin, promoter of mucin-like glycoprotein (MUC1), promoter of prostate specific antigen (PSA), promoter of survivin, promoter of tyrosinase related protein (TRP1), and promoter of tyrosinase.

In certain embodiments, the promoters of the present disclosure may be tumor specific promoters. The term “tumor specific promoter” as used herein refers to a promoter that functions to activate gene expression preferentially or exclusively in tumor cells, and has no activity or reduced activity in non-tumor cells or non-cancer cells. Illustrative examples of tumor specific promoters include, without limitation, E2F-1 promoter, promoter of alpha-fetoprotein, promoter of cholecystokinin, promoter of carcinoembryonic antigen, promoter of C-erbB2/neu oncogene, promoter of cyclooxygenase, promoter of CXCR4, promoter of HE4, promoter of hexokinase type II, promoter of L-plastin, promoter of MUC1, promoter of PSA, promoter of survivin, promoter of TRP1, and promoter of tyrosinase. In certain embodiments, the promoter of the present disclosure is E2F-1. In certain embodiments, the E2F-1 promoter has a nucleotide sequence of SEQ ID NO: 29.

The first promoter and/or second promoter described above may be a constitutive promoter. In certain embodiments, the constitutive promoter can be a promoter of house-keeping genes which are required for fundamental cellular functions. Illustrative examples include, without limitation, promoter of beta-actin promoter, UBC promoter, PGK promoter, TK promoter, and EF1A promoter. In certain embodiments, the constitutive promoter can be a viral promoter. Illustrative examples include, without limitation, CMV promoter, SV40 promoter, and CaMV 35S promoter.

In certain embodiments, the recombinant nucleic acid composition of the present disclosure contains a first polynucleotide encoding an antigen presenting polypeptide that is controlled by a constitutive promoter such as CMV promoter, and a second polynucleotide encoding a cytokine that is controlled by a tumor specific promoter such as E2F-1 promoter.

In certain embodiments, the antigen presenting polypeptides are heat shock proteins. The heat shock proteins are a family of proteins whose expression are increased when cells are exposed to elevated temperatures or other stress. Heat shock proteins are known to play an important role in folding the proteins to make a proper conformation. In addition, heat shock proteins are found to present protein fragments to the immune system.

Any heat shock protein may be used as the antigen presenting polypeptide for the present disclosure. Illustrative examples of heat shock proteins include, without limitation, Hsp10, Hsp20, Hsp27, Hsp40, Hsp60, Hsp70, Hsp71, Hsp72, Hsp90, Hsp104, Hsp110, alpha-B-crystallin, and glucose-regulated proteins.

In certain embodiments, the antigen presenting polypeptide is Hsp70. Hsp70 proteins include, without limitation, Hsp70-1, Hsp70-2, Hsp70-4, Hsp70-5, Hsp70-6, Hsp70-7, Hsp70-8, Hsp70-9, Hsp70-12, and Hsp70-14. In certain embodiments, the antigen presenting polypeptide is Hsp70-1. Hsp70-1 proteins include, without limitation, Hsp 70-1A, and Hsp 70-1B.

In certain embodiments, the antigen presenting polypeptide is Hsp90. Hsp90 proteins include, without limitation, Hsp90-α₁, Hsp90-α₂, Hsp90-β, endoplasmin, and TNF receptor-associated protein 1.

In certain embodiments, the antigen presenting polypeptide is glucose-regulated protein. The glucose-regulated protein include, without limitation, glucose-regulate protein 75 (Grp75, also known as Hsp70-9), Grp78 (also known as Hsp70-5), Grp94 (also known as endoplasmin), and Grp170.

Illustrative examples of heat shock proteins and their GenBank accession numbers are listed in Table 1.

TABLE 1
Examples of heat shock proteins

| | | Nucleotide sequence | | Peptide sequence | |
|---|---|---|---|---|---|
| | | GenBank | SEQ ID | GenBank | SEQ ID |
| Species | Name | Accession No. | NOs | Accession No. | NOs |
| Homo sapiens | HSP70 | NM_005345.5 | SEQ ID | NP_005336.3 | SEQ ID |
| (Human) | 1A | | NO: 1 | | NO: 2 |
| Homo sapiens | Hsp70-4 | NM_002154.3 | SEQ ID | NP_002145.3 | SEQ ID |
| (Human) | | | NO: 3 | | NO: 4 |
| Homo sapiens | Hsp90 α | NM_001017963.2 | SEQ ID | NP_001017963.2 | SEQ ID |
| (Human) | isoform 1 | | NO: 5 | | NO: 6 |
| Homo sapiens | Grp-75 | NM_004134.6 | SEQ ID | NP_004125.3 | SEQ ID |
| (Human) | | | NO: 7 | | NO: 8 |
| Mus musculus | Grp-78 | NM_022310.3 | SEQ ID | NP_071705.3 | SEQ ID |
| (house mouse) | | | NO: 9 | | NO: 10 |
| Rattus norvegicus | alpha- | NM_012935.3 | SEQ ID | NP_037067.1 | SEQ ID |
| (Norway rat) | crystallin | | NO: 11 | | NO: 12 |
| | B chain | | | | |

In certain embodiments, the antigen presenting polypeptides of the present disclosure is a fragment of a full length heat shock protein that retains the function of presenting antigens to the antigen presenting cells. In certain embodiments, the antigen presenting polypeptides of the present disclosure is a functional equivalent of a naturally occurring antigen presenting polypeptide. The functional equivalent is structurally homologous to the naturally occurring antigen presenting polypeptide and can function to present antigens to the antigen presenting cells. A functional equivalent of a naturally occurring antigen presenting polypeptide may have one or more conservative or non-conservative amino acid substitutions, additions, deletions, modifications or other changes to a naturally occurring antigen presenting polypeptide sequence.

In certain embodiments, the antigen presenting polypeptides of the present disclosure share peptide sequence identity with one or more heat shock proteins. The term “peptide sequence identity” as used herein refers to the amino acid residues in a candidate sequence that are identical with the amino acid residues in the template sequence, after aligning the sequences and introducing gaps, if necessary, to achieve the maximum number of identical amino acid residues between the candidate sequence and the template sequence, and not considering any conservative substitutions as part of the sequence identity.

Identity of two amino acid sequences may be determined by aligning the two amino acid sequences. Publicly available computer softwares may be used in the alignment, such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In certain embodiments, the antigen presenting polypeptide has at least 50%, or at least 60%, or at least 70%, or at least 80%, or at least 90%, or at least 95%, or at least 97%, or at least 99% peptide sequence identity with one of Hsp10, Hsp20, Hsp27, Hsp40, Hsp60, Hsp70, Hsp71, Hsp72, Hsp90, Hsp104, Hsp110, alpha-B-crystallin, and glucose-regulated proteins.

In certain embodiments, the antigen presenting polypeptide has at least 50%, or at least 60%, or at least 70%, or at least 80%, or at least 90%, or at least 95%, or at least 97%, or at least 99% peptide sequence identity with Hsp70.

In certain embodiments, the antigen presenting polypeptide contains one or more functional domains from one or more heat shock proteins. The term “functional domain” as used herein refers to a part of a heat shock protein that has the function of presenting antigens to the antigen presenting cells, which function is independent of the rest of the protein sequence and structure. In certain embodiments, the antigen presenting polypeptide contains one or more functional domains derived from one or more heat shock proteins selected from the group consisting of Hsp10, Hsp27, Hsp40, Hsp60, Hsp70, Hsp71, Hsp72, Hsp90, Hsp104, alpha-B-crystallin, and glucose-regulated protein.

In certain embodiments, the nucleic acid composition contains one or more polynucleotides encoding one or more heat shock proteins. In certain embodiments, a polynucleotide of the present disclosure may encode for one, two, three or four heat shock proteins. In certain embodiments, the composition of the present disclosure contains one, two, three, or four polynucleotides, each of which encodes for a heat shock protein. In certain embodiments, the composition contains a polynucleotide encoding for two or more heat shock proteins.

The term “cytokine” as used herein refers to a protein or polypeptide that is secreted by cells and has immunomodulating effects, such as, stimulating the functions of immune cells, promoting growth of immune cells, or directing immune cells to a local site in need.

Any suitable cytokines may be used for the present disclosure. Illustrative examples of cytokines include, without limitation, colony-stimulating factors such as granulocyte macrophage colony-stimulating factors (GM-CSFs), macrophage-colony stimulating factors (M-CSFs), granulocyte-colony stimulating factors (G-CSFs), stem cell factors, and erythropoietin; interferons (IFNs) such as IFN α, IFN β, IFN γ, and IFN ω; interleukins such as IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, and IL-35; tumor necrosis factors (TNFs) such as TNF α, and TNF β; chemokines such as CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, and CX3L1.

In certain embodiments, the cytokines of the present disclosure can stimulate the function and/or accumulation of antigen presenting cells. In certain embodiments, the cytokines include M-CSFs, G-CSFs, GM-CSFs, stem cell factors, and erythropoietin. In certain embodiments, the cytokines of the present disclosure are GM-CSFs. Illustrative examples of GM-CSFs and their GenBank accession numbers are listed in Table 2.

TABLE 2
Examples of GM-CSFs

| | | Nucleotide sequence | | Peptide sequence | |
|---|---|---|---|---|---|
| | | GenBank | SEQ ID | GenBank | SEQ ID |
| Species | Name | Accession No. | NOs | Accession No. | NOs |
| Homo sapiens | GM-CSF | M11220.1 | SEQ ID | AAA52578.1 | SEQ ID |
| (Human) | | | NO: 13 | | NO: 14 |
| Homo sapiens | GM-CSF | BC113924.1 | SEQ ID | AAI13925.1 | SEQ ID |
| (Human) | | | NO: 15 | | NO: 16 |
| Mus musculus | GM-CSF | X03019.1 | SEQ ID | CAA26820.1 | SEQ ID |
| (house mouse) | | | NO: 17 | | NO: 18 |
| Mus musculus | GM-CSF | EU366957.1 | SEQ ID | ABY66391.1 | SEQ ID |
| (house mouse) | | | NO: 19 | | NO: 20 |
| Felis catus | GM-CSF | AF053007.1 | SEQ ID | AAC06041.1 | SEQ ID |
| (domestic cat) | | | NO: 21 | | NO: 22 |
| Rattus norvegicus | GM-CSF | U00620.1 | SEQ ID | AAA18281.1 | SEQ ID |
| (Norway rat) | | | NO: 23 | | NO: 24 |
| Homo sapiens | GM-CSF | AC004511.1 | SEQ ID | AAC08707.1 | SEQ ID |
| (Human) | | | NO: 25 | | NO: 26 |

In certain embodiments, the cytokine of the present disclosure is a fragment of a cytokine that retains its function of immunomodulating effects.

In certain embodiments, the cytokines of the present disclosure share peptide sequence identity with one or more cytokines. In certain embodiments, the cytokine encoded by the second polynucleotide has at least 50%, or at least 60%, or at least 70%, or at least 80%, or at least 90%, or at least 95%, or at least 97%, or at least 99% peptide sequence identity with one or more of M-CSFs, G-CSFs, GM-CSFs, stem cell factors, and erythropoietin.

In certain embodiments, the cytokine encoded by the second polynucleotide has at least 50%, or at least 60%, or at least 70%, or at least 80%, or at least 90%, or at least 95%, or at least 97%, or at least 99% peptide sequence identity with GM-CSFs.

In certain embodiments, the cytokine contains one or more functional domains from one or more cytokines. In certain embodiments, the antigen presenting polypeptide contains one or more functional domains derived from one or more cytokines selected from the group of M-CSFs, G-CSFs, GM-CSFs, stem cell factors, and erythropoietin.

In certain embodiments, the nucleic acid composition contains one or more polynucleotides encoding one or more cytokines. In certain embodiments, a polynucleotide of the present disclosure may encode for one, two, three or four cytokines. In certain embodiments, the composition of the present disclosure contains one, two, three, or four polynucleotides, each of which encodes for a cytokine. In certain embodiments, the composition contains a polynucleotide encoding for two or more cytokines.

In certain embodiment, the composition further contains a third polynucleotide encoding for a cell toxic gene. The term “cell toxic gene” as used herein refers to a gene that can function to induce or enhance cytotoxicity including cell death. In certain embodiments, a cell toxic gene can improve cell susceptibility to lysis by other cells such as macrophages. Illustrative examples of the cell toxic genes include, without limitation, thymidine kinase gene, and cytosine deaminase gene.

The third polynucleotide may be operably linked to a promoter. In certain embodiments, the expression of the first polynucleotide, the second polynucleotide and the third polynucleotide is controlled by the same promoter. In certain embodiments, the expression of two of the first, second and third polynucleotide is controlled by the same promoter. In certain embodiments, one or more IRES sequence are used to separate the encoding sequences on the same polynucleotide molecule such that the encoded products can be separately translated and expressed from the polynucleotide molecule.

In certain embodiments, different promoters control the expression of the first polynucleotide, the second polynucleotide and the third polynucleotide. In certain embodiments, the first polynucleotide is operably linked to a first promoter, the second polynucleotide is operably linked to a second promoter, and the third polynucleotide is operably linked to a third promoter. In certain embodiments, the first, second and/or third promoters are eukaryotic promoters. In certain embodiments, the first, second and/or third promoters are constitutive promoters. In certain embodiments, the first, second and/or third promoters are tumor specific promoters. In certain embodiments, at least one of the first, second and third promoters are tumor specific promoters. In certain embodiments, at least two of the first, second and third promoters are tumor specific promoters. In certain embodiments, all of the first, second and third promoters are tumor specific promoters.

The nucleic acid sequences of the present disclosure are available publicly or may be readily obtained by a person skilled in the art. The polynucleotides or nucleic acids of the present disclosure can be made through well known recombinant techniques in the art (see, for example, Sambrook et al. Molecular Cloning: A Laboratory Manual (Cold Spring Harbor Laboratory, N.Y. (1989), which is incorporated herein by reference in its entirety).

The Expression Vector

The polynucleotides described above may be inserted into one or more expression vectors. The present disclosure provides an expression vector containing a first polynucleotide segment encoding an antigen presenting polypeptide, a second polynucleotide segment encoding a cytokine, and at least one promoter, wherein the at least one promoter is operably linked to at least one of the first polynucleotide segment and the second polynucleotide segment.

The term “expression vector” as used herein refers to a polynucleotide vehicle capable of transforming or transfecting cells and allowing gene expression in the cells. The term “segment” as used herein refers to a portion or a fragment of a nucleic acid sequence.

The expression vectors of the present disclosure may be viral vectors, plasmids, phages or cosmids. In certain embodiments, the expression vectors of the present disclosure may further comprise one or more gene sequences derived from a virus, a plasmid, a phage or a cosmid or fragments thereof.

In certain embodiments, the expression vectors of the present disclosure are viral vectors. Viral vectors can contain the whole or a portion of a viral genome. In certain embodiment, a viral vector contains the whole or a portion of the viral genome of two or more viruses.

In certain embodiments, the viral vector is derived from an adenovirus. Adenovirus has a double-strand linear DNA genome which can not be integrated into the host genome. Illustrative examples of adenovirus vectors include, without limitation, first generation adenovectors (e.g., adenovector having E1a and E1b genes deleted, and adenovector having E1 and E3 genes deleted), second generation adenoviral vectors (e.g. adenovector having E1 and E2 genes deleted, adenovector having E1 and E4 genes deleted), and gutless adenoviral vectors in which all viral coding sequences are deleted (also called helper dependent adenoviral vector).

In certain embodiments, the viral vector is derived from an adeno-associated virus (AAV). Adeno-associated virus can infect both dividing and non-dividing cells and can integrate its genome to that of the host cells. Illustrative examples of adeno-associated virus vectors include, without limitation, vectors derived from AAV serotype 1, AAV serotype 2, AAV serotype 3, AAV serotype 4, AAV serotype 5, AAV serotype 6, AAV serotype 7, AAV serotype 8, AAV serotype 9, AAV serotype 10, AAV serotype 11, and AAV serotype 12.

In certain embodiments, the viral vector is derived from a retrovirus. A retrovirus has a RNA genome and can replicate in a host cell via reverse transcriptase to produce DNA from the RNA genome. Illustrative examples of retrovirus vectors include, without limitation, vectors derived from avian leucosis virus, mouse mammary tumour virus, murine leukaemia virus, bovine leukaemia virus, Walleye dermal sarcoma virus, HIV-1 (Human Immunodeficiency Virus), HIV-2, SIV (simian immunodeficiency virus), EIAV (equine infectious anaemia virus), FIV (feline immunodeficiency virus), CAEV (caprine arthritis encephalitis virus), VMV (visna/maedi virus), human spumavirus, moloney murine leukaemia virus, rous sarcoma virus, feline leukemia virus, human T-lymphotropic virus, and simian foamy virus.

In certain embodiments, the viral vector is derived from a lentivirus. Illustrative examples of lentivirus vectors include, without limitation, vectors derived from HIV-1, SIV, FIV, CAEV, VMV, and EIAV.

In certain embodiments, the viral vector is derived from an alphavirus. Illustrative examples of alphavirus vectors include, without limitation, vectors derived from venezuelan equine encephalitis virus, sindbis virus, semliki forest virus, eastern equine encephalitis virus, western equine encephalitis virus, O'nyong'nyong virus, chikungunya virus, mayaro virus, ross river virus, and barmah forest virus.

In certain embodiments, the vector is derived from a poxvirus. Illustrative examples of poxvirus vectors include, without limitation, vectors derived from vaccinia virus, variola virus, vaccinia virus, avipox virus, cowpox virus, monkeypox virus, smallpox, orf virus, pseudocowpox, bovine papular stomatitis virus, tanapox virus, yaba monkey tumor virus, and molluscum contagiosum virus.

In certain embodiments, the vector is derived from a herpesvirus. Illustrative examples of Herpesvirus vectors include, without limitation, vectors derived from herpes simplex virus-1 (HSV-1), herpes simplex virus-2, varicella zoster virus, Epstein-Barr virus (EBV), lymphocryptovirus, CMV, herpes lymphotropic virus, roseolovirus, and Kaposi's sarcoma-associated herpesvirus (KSHV).

In certain embodiments, the vector is derived from a poliovirus. Illustrative examples of poliovirus vectors include, without limitation, vectors derived from poliovirus serotype 1 (PV1), PV2, and PV3.

In certain embodiments, the expression vector is derived from a baculovirus. Illustrative examples of baculovirus vectors include, without limitation, expression vectors derived from autographa californica multicapsid nucleopolyhedrovirus (AcMNPV), orgyia pseudotsugata multicapsid polyhedrosis virus (OpMNPV), lymantria dispar multicapsid nuclear polyhedrosis virus (LdMNPV), bombyx mori nuclear polyhedrosis virus (BmNPV), choristoneura fumiferana nuclear polyhedrosis virus (CfMNPV), cryptophlebia leucotreta granulosis virus (ClGV), heliothis zea nuclear polyhedrosis virus (HzSNPV), and cydia pomonella granulosis virus (CpGV).

In certain embodiments, the expression vector is derived from a papillomavirus. Illustrative examples of papillomavirus vectors include, without limitation, vectors derived from bovine papillomavirus type 4, bovine papillomavirus type 2, human papillomavirus type 11, human papillomavirus type 5b, human papillomavirus type 6b, bovine papillomavirus type 1, cottontail rabbit papillomavirus (CRPV), deer papillomavirus (DPV), human papillomavirus type 13, and human papillomavirus type 16.

In certain embodiments, the expression vector is derived from a polyomavirus. Illustrative examples of the polyomavirus vector include, without limitation, vectors based on budgerigar fledgling disease virus (BFPyV), simian virus 40 (SV40), BK polyomavirus (BKPyV), bovine polyomavirus (BPyV), hamster polyomavirus (HaPyV), JC polyomavirus (JCPyV) (JCV), murine polyomavirus (MPyV), and B-lymphotropic polyomavirus (LPV).

In certain embodiments, the expression vector is derived from a parvovirus. Illustrative examples of parvovirus vectors include, without limitation, expression vectors derived from canine parvovirus, feline panleukopenia virus (FPV), hamster parvovirus H1, mink enteritis virus (MEV), murine minute virus (MVM), parvovirus LuIII, and porcine parvovirus (PPV).

In certain embodiments, the viral vector is derived from a measles virus. In certain embodiments, the viral vector is derived from a vesicular stomatitis virus (VSV). In certain embodiments, the viral vector is derived from a respiratory and enteric orphan virus (REO virus). In certain embodiments, the viral vector is derived from a Newcastle disease virus. In certain embodiments, the viral vector is derived from a Sendai virus (SeV).

In certain embodiments, the viral vector is a vector derived from one or more viruses selected from the group consisting of adenovirus, retrovirus, lentivirus, vaccinia virus, HSV, VSV, measles virus, REO virus, Newcastle disease virus, and SeV.

In certain embodiments, the viral vector is capable of cell lysis. Cell-lytic viral vectors can replicate in the host cells, and assemble into viral particles which are released from the cell by lysis of the host cell. Illustrative examples of cell-lytic virus vectors include, without limitation, vectors derived from adenovirus, retrovirus, lentivirus, vaccinia virus, HSV, VSV, measles virus, REO virus, Newcastle disease virus, and SeV.

In certain embodiments, the viral vector contains a tumor specific promoter for expression of the viral genes upon infection of tumor cells.

In certain embodiments, the expression vector is a plasmid. Illustrative examples of the plasmid vector include, without limitation, pBR322, pTZ19R, pUC18, pUC19, pUC57, pCMV-Script system, and pcDNA3.

In certain embodiments, the expression vector is a phage vector. Illustrative examples of the phages include, without limitation, T4 phage, lambda phage, T7 phage, p1 phage, pBlueScript II phagemid, and pBC phagemid.

In certain embodiments, the expression vector is a cosmid vector. Illustrative examples of the cosmid vector include, without limitation, SuperCos 1 vector, pJC81, and pHS262.

The expression vectors of the present disclosure may be transient expression vectors that do not integrate into the genome of the host cells or stable expression vectors that can integrate its genes into the genome of a host cell after introduction into the host cell.

In certain embodiments, the expression vector is a transient expression vector. As the host cell divides, the transient expression vectors are diluted in the host cells, and thus provide expression of exogenous genes only for a limited time. Illustrative examples of transient expression vectors include, without limitation, adenovirus vectors, herpesvirus vectors, poxvirus vectors, non-integrating plasmids or phages.

In certain embodiments, the expression vector is a stable expression vector. The stable expression vector can integrate into the host genome at any random sites or at certain designated sites. The integrated genes can replicate along with the host genome, and thus allow stable expression of the genes in the host cells. Illustrative examples of stable expression vectors include, without limitation, vectors derived from retrovirus, lentivirus, adeno-associated virus, and integrating plasmids.

The expression vectors of the present disclosure can be replication competent vectors or replication defective vectors. In certain embodiments, the expression vectors are replication competent vectors. The term “replication competent vector” as used herein refers to a vector that contains the sequence(s) necessary for self replication and can replicate by itself (without integrating into the host genome) in a host cell. In certain embodiments, the replication competent vectors can selectively or conditionally replicate in certain specific host cells, such as, tumor cells.

In certain embodiments, the replication competent vectors may be virus vectors. Illustrative examples of replication competent virus vectors include, without limitation, vectors derived from adenovirus, vaccinia virus, HSV, VSV, measles virus, REO virus, Newcastle disease virus and SeV. In certain embodiments, the replication competent viral vectors contain viral genes essential for virus replication. For example, a replication competent adenovirus vector contains the E1a gene of the adenovirus. In certain embodiments, the E1a gene is operably linked to a tumor specific promoter. In certain embodiments, the E1a gene of adenovirus has a nucleotide sequence of SEQ ID NO: 30.

In certain embodiments, the expression vector can be a replication defective vector. The term “replication defective vector” as used herein refers to a vector that cannot replicate by itself in a host cell. In certain embodiments, the replication defective vector can be a virus vector. Illustrative examples of replication defective virus vectors include, without limitation, adenovirus vectors that lack or are defective in E1a gene function.

In certain embodiments, an expression vector of the present disclosure contains a first polynucleotide segment encoding for an antigen presenting peptide, where the antigen presenting peptide is a heat shock protein. In certain embodiments, the expression vector further contains a second polynucleotide segment encoding for a cytokine, where the cytokine is a GM-CSF. In certain embodiments, the expression vector contains one or more promoters operably linked to the first and/or second polynucleotide segments.

In certain embodiments, the promoters operably linked to the first and/or second polynucleotide segments of the expression vector are eukaryotic promoters. In certain embodiments, the promoters are constitutive promoters such as the beta-actin promoter, UBC promoter, PGK promoter, TK promoter, EF1A promoter, CMV promoter, SV40 promoter, and CaMV 35S promoter. In certain embodiments, the promoters are tumor specific promoters such as the E2F-1 promoter, promoter of alpha-fetoprotein, promoter of cholecystokinin, promoter of carcinoembryonic antigen, promoter of C-erbB2/neu oncogene, promoter of cyclooxygenase, promoter of CXCR4, promoter of HE4, promoter of hexokinase type II, promoter of L-plastin, promoter of MUC1, promoter of PSA, promoter of survivin, promoter of TRP1, and promoter of tyrosinase.

In certain embodiments, the expression of the first polynucleotide segment and the second polynucleotide segment is controlled by the same promoter. In certain embodiments, the first polynucleotide segment and the second polynucleotide segment are operably linked to one promoter, in which the first polynucleotide segment and the second polynucleotide segment are separated by an IRES sequence.

In certain embodiments, the expression of the first polynucleotide segment and the second polynucleotide segment are controlled by different promoters. In certain embodiments, the first polynucleotide segment is operably linked to a first promoter, and the second polynucleotide segment is operably linked to a second promoter. In certain embodiments, the first promoter is a constitutive promoter and the second promoter is a tumor specific promoter. In certain embodiments, the first promoter is a tumor specific promoter and the second promoter is a constitutive promoter. In certain embodiments, the first and second promoters are both tumor specific promoters.

In certain embodiment, the expression vector further contains a third polynucleotide segment encoding for a cell toxic gene. The third polynucleotide segment may be operably linked to a promoter. In certain embodiments, the expression of the first polynucleotide segment, the second polynucleotide segment and the third polynucleotide segment is controlled by the same promoter. In certain embodiments, the expression of at least two of the first, second and third polynucleotide segments is controlled by the same promoter. In certain embodiments, the expression of the first polynucleotide segment and the third polynucleotide segment is controlled by the same promoter. In certain embodiments, the expression of the second polynucleotide segment and the third polynucleotide segment is controlled by the same promoter. In certain embodiments, one or more IRES sequences are used to separate the encoding sequences on the same polynucleotide molecule such that the encoded products can be separately translated and expressed from the polynucleotide molecule.

In certain embodiments, the expression of the first polynucleotide segment, the second polynucleotide segment and the third polynucleotide segment is controlled by different promoters. In certain embodiments, the first polynucleotide segment is operably linked to a first promoter, the second polynucleotide segment is operably linked to a second promoter, and the third polynucleotide segment is operably linked to a third promoter. In certain embodiments, the first, second and/or third promoter is a eukaryotic promoter. In certain embodiments, the first, second and/or third promoter is a constitutive promoter. In certain embodiments, the first, second and/or third promoter is a tumor specific promoter.

The expression vectors can be produced using any suitable recombinant techniques, such as without limitation, molecular cloning techniques involving the use of restriction enzymes and nucleic acid ligation, and homologous recombination techniques involving exchange of nucleotide sequences between two similar nucleic acid strands (for review please see, Sambrook et al., Molecular Cloning, A Laboratory Manual, 3rd Edition, Cold Spring Harbor Laboratory Press, 2001; C. K. Raymond et al, Biotechniques, 26(1): 134-141 (1999)). For example, a commercially available vector may be linearized using restriction enzymes, and then ligated with a target sequence (e.g. the first polynucleotide and/or the second polynucleotide and/or the tumor specific promoter). The target sequence may be obtained through PCR reaction using primers complementary to the target sequence, and templates containing the target sequence isolated and purified from cells containing such target sequence. The target sequence may also be obtained by digestion from a readily available vector using restriction enzymes generating corresponding restriction sites. After ligation, the obtained vector may be transformed into a suitable host cell, and the host cell containing the ligated vector is selected in a selection medium which contains a selection marker (e.g. antibiotics). The selected host cells are propagated, and the vectors may be isolated and purified from the host cells using well known methods such as ethanol precipitation or gel extraction. The obtained vector may be subsequently inserted with another sequence of interest using similar methods, or may be digested with restriction enzymes to generate another target sequence for further use, or may be used in homologous recombination with another vector having similar sequences.

The expression vectors of the present invention may be used to transfect host cells for expression of the encoded sequences. In another aspect, the present disclosure provides host cells containing the expression vectors provided herein. Any suitable cells, to which the expression vector may be transfected and replicated therein, may be used as the host cell. Illustrative examples of host cells include without limitation, yeast cells and mammalian cells. The host cells may also be cells taken from a subject in need of treatment using a treatment method of the present disclosure as described below. The expression vectors may be introduced into the host cells using any suitable methods known in the art, including, without limitation, electroporation, calcium chloride-, lithium chloride-, lithium acetate/polyethylene glycol-, calcium phosphate-, DEAE-dextran-, liposome-mediated polynucleotide uptake, spheroplasting, injection, microinjection, microprojectile bombardment, phage infection, viral infection, or other established methods. Alternatively, the expression vectors containing the gene sequences of interest can be transcribed in vitro, and the resulting mRNA may be introduced into the host cell for transient expression by well-known methods, e.g., by injection (see, Kubo et al., FEBS Letts. 241: 119, (1988)). For example, the expression vector may be mixed with a calcium chloride solution and then combined with a HEPES-buffered saline, forming fine precipitate of calcium phosphate that binds to the expression vector, upon introduction to the cell culture, the expression vector is taken up by the cells together with the precipitate. For another example, the expression vector may be encapsulated in liposome bodies and introduced to cells, when the liposomes fuse with the cell membrane, they release the encapsulated expression vector into the cells. For yet another example, the expression vector may be delivered into cells under an electric discharge which creates micro-holes in the cell membrane. For still another example, the expression vector may be coupled to a nanoparticle of an inert solid (e.g. gold particles) and then shot directly into the target cell's nucleus using a specialized equipment such as a gene gun. For still another example, the expression vector itself may be a virus which can infect the cells and releases the polynucleotide materials into the cells.

Host cells containing the expression vectors provided herein can be provided in any suitable forms. In an illustrative embodiment, the host cells are isolated from cell cultures, where the cell cultures may be filtered or centrifuged or otherwise treated to remove the culture media, cell debris and/or other unwanted substances. The host cells may undergo further purification processes as deemed suitable by a person skilled in the art to increase the concentration of the expression vectors therein. The composition may be prepared in liquid form or dried solid form.

Pharmaceutical Composition

The nucleic acid compositions, expression vectors, and host cells described in the present disclosure may be used as tumor vaccines to prevent and/or treat tumors and/or cancers. In another aspect, the present disclosure provides a pharmaceutical composition comprising a pharmaceutically acceptable carrier and the nucleic acid composition, expression vectors, and/or host cells provided herein. The nucleic acids, expression vectors and host cells can be constructed, isolated and purified using methods well known in the art. The term “pharmaceutically acceptable carrier” as used herein refers to any and all solvents, dispersion media, coatings, antibacterial and antifungal agents, isotonic and absorption delaying agents, and the like that can facilitate storage and administration of the nucleic acids, expression vectors, and/or host cells of the present disclosure to a subject. The pharmaceutically acceptable carriers can include any suitable components, such as without limitation, saline, liposomes, polymeric excipients, colloids, or carrier particles.

In certain embodiments, the pharmaceutically acceptable carriers are saline that can dissolve or disperse the nucleic acids, expression vectors, and/or host cells of the present disclosure. Illustrative examples of saline include, without limitation, buffer saline, normal saline, phosphate buffer, citrate buffer, acetate buffer, bicarbonate buffer, sucrose solution, salts solution and polysorbate solution.

In certain embodiments, the pharmaceutically acceptable carriers are liposomes. Liposomes are uni-lamellar or multi-lamellar vesicles, having a membrane formed of lipophilic material and an interior aqueous portion. The nucleic acids, expression vectors, and/or host cells of the present disclosure can be encapsulated within the aqueous portion of the liposomes. Illustrative examples of liposomes include, without limitation, liposomes based on 3[N—(N′,N′-dimethylaminoethane)carbamoyl] cholesterol (DC-Chlo), liposomes based on N-(2,3-dioleoyloxy)propyl-N,N,N-trimethylammonium chloride (DOTMA), and liposomes based on 1,2-dioleoyloxy-3-trimethylammonium propane (DOTAP). Methods of preparing liposomes and encapsulating the expression vectors in liposomes are well known in the art (see for example, D. D. Lasic et al, Liposomes in gene delivery, published by CRC Press, 1997).

In certain embodiments, the pharmaceutically acceptable carriers are polymeric excipients, such as without limitation, microspheres, microcapsules, polymeric micelles and dendrimers. The nucleic acids, expression vectors, and/or host cells of the present disclosure may be encapsulated, adhered to, or coated on the polymer-based components by methods known in the art (see for example, W. Heiser, Nonviral gene transfer techniques, published by Humana Press, 2004; U.S. Pat. No. 6,025,337; Advanced Drug Delivery Reviews, 57(15): 2177-2202 (2005)).

In certain embodiments, the pharmaceutically acceptable carriers are colloids or carrier particles such as gold colloids, gold nanoparticles, silica nanoparticles, and multi-segment nanorods. The nucleic acids, expression vectors or cells may be coated on, adhered to, or associated with the carriers in any suitable manner as known in the art (see for example, M. Sullivan et al., Gene Therapy, 10: 1882-1890 (2003), C. McIntosh et al., J. Am. Chem. Soc., 123 (31): 7626-7629 (2001), D. Luo et al., Nature Biotechnology, 18: 893-895 (2000), and A. Salem et al., Nature Materials, 2: 668-671 (2003)).

In certain embodiments, the pharmaceutical composition may further comprise additives, such as without limitation, stabilizers, preservatives, and transfection facilitating agents which assist in the cellular uptake of the medicines. Suitable stabilizers may include, without limitation, monosodium glutamate, glycine, EDTA and albumin. Suitable preservatives may include, without limitation, 2-phenoxyethanol, sodium benzoate, potassium sorbate, methyl hydroxybenzoate, phenols, thimerosal, and antibiotics. Suitable transfection facilitating agents may include, without limitation, calcium ions.

In certain embodiments, the pharmaceutical composition further comprises one or more anti-tumor agents. Any agents known to be active against tumor or cancer may be used in the composition. In certain embodiments, the anti-tumor agent is selected from the group consisting of a chemical agent, a polynucleotide, a peptide, a protein, or any combination thereof.

In certain embodiments, the anti-tumor agent is a chemical agent. Illustrative examples of anti-tumor chemical agent include, without limitation, Mitomycin C, Daunorubicin, Doxorubicin, Etoposide, Tamoxifen, Paclitaxel, Vincristine, and Rapamycin.

In certain embodiments, the anti-tumor agent is a polynucleotide. Illustrative examples of anti-tumor polynucleotide include, without limitation, anti-sense oligonucleotides such as bcl-2 antisense oligonucleotides, clusterin antisense oligonucleotides, and c-myc antisense oligonucleotides; and RNAs capable of RNA interference (including small interfering RNA (siRNA), short hairpin RNA (shRNAs), and micro interfering RNAs (miRNA)), such as anti-VEGF siRNA, shRNA, or miRNA, anti-bcl-2 siRNA, shRNA, or miRNA, and anti-claudin-3 siRNA, shRNA, or miRNA.

In certain embodiments, the anti-tumor agent is a peptide or protein. Illustrative examples of anti-tumor peptide or protein include, without limitation, antibodies such as, Trastuzumab, Rituximab, Edrecolomab, Alemtuzumab, Daclizumab, Nimotuzumab, Gemtuzumab, Ibritumomab, and Edrecolomab, protein theraputics such as, Endostatin, Angiostatin K1-3, Leuprolide, Sex hormone-binding globulin, and Bikunin.

In another aspect, the present disclosure provides a tumor vaccine containing a first polynucleotide encoding an antigen presenting polypeptide, a second polynucleotide encoding a cytokine, and at least one promoter, where the at least one promoter is operably linked to at least one of the first polynucleotide and the second polynucleotide.

In another aspect, the present disclosure provides a tumor vaccine containing a first polynucleotide encoding an antigen presenting polypeptide, a second polynucleotide encoding a cytokine, where the first polynucleotide is operably linked to a constitutive promoter such as the CMV promoter, and the second polynucleotide is operably linked to a tumor specific promoter such as the E2F-1 promoter of adenovirus.

In another aspect, the present disclosure provides a tumor vaccine containing an expression vector containing a first polynucleotide segment encoding an antigen presenting polypeptide, a second polynucleotide segment encoding a cytokine, and at least one promoter, where the at least one promoter is operably linked to at least one of the first polynucleotide and the second polynucleotide.

In another aspect, the present disclosure provides a tumor vaccine containing an expression vector containing a first polynucleotide segment encoding an antigen presenting polypeptide, a second polynucleotide segment encoding a cytokine, where the first polynucleotide segment is operably linked to a constitutive promoter such as the CMV promoter, and the second polynucleotide segment is operably linked to a tumor specific promoter such as the E2F-1 promoter of adenovirus.

In another aspect, the present disclosure provides a tumor vaccine having a host cell containing an expression vector containing a first polynucleotide segment encoding an antigen presenting polypeptide, a second polynucleotide segment encoding a cytokine, and at least one promoter, where the at least one promoter is operably linked to at least one of the first polynucleotide and the second polynucleotide.

In another aspect, the present disclosure provides a tumor vaccine having a host cell containing an expression vector containing a first polynucleotide segment encoding an antigen presenting polypeptide, a second polynucleotide segment encoding a cytokine, where the first polynucleotide segment is operably linked to a constitutive promoter such as the CMV promoter, and the second polynucleotide segment is operably linked to a tumor specific promoter such as the E2F-1 promoter of adenovirus.

In another aspect, the present disclosure provides a tumor vaccine having virus particles wherein the virus genome contains a first polynucleotide segment encoding an antigen presenting polypeptide, a second polynucleotide segment encoding a cytokine. In certain embodiments, the first polynucleotide segment of the virus genome is operably linked to a constitutive promoter such as the CMV promoter, and the second polynucleotide segment of the virus genome is operably linked to a tumor specific promoter such as the E2F-1 promoter of adenovirus. In certain embodiments, the virus particles are recombinant adenovirus.

In certain embodiments, the tumor vaccine further comprises an adjuvant. The term “adjuvant” as used herein refers to an agent that, when administered with the vaccine composition provided herein, can non-specifically enhance the immune response to the vaccine.

Method of Treatment

In another aspect, the present disclosure provides a method of treating a tumor, comprising administering into a subject a pharmaceutical composition containing a pharmaceutically acceptable carrier and the tumor vaccine of the present disclosure.

The term “subject” as used herein, includes animals and humans.

The pharmaceutical composition may be administered via any suitable routes known in the art, including without limitation, parenteral, oral, enteral, buccal, nasal, topical, rectal, vaginal, transmucosal, epidermal, transdermal, dermal, ophthalmic, pulmonary, and subcutaneous administration routes.

The pharmaceutical composition can be administered to a subject in the form of formulations or preparations suitable for each administration route. Formulations suitable for administration of the pharmaceutical composition may include, without limitation, solutions, dispersions, emulsions, powders, suspensions, aerosols, sprays, nose drops, liposome based formulations, patches, implants and suppositories.

The formulations may conveniently be presented in unit dosage form and may be prepared by any methods well known in the art of pharmacy. Methods of preparing these formulations or compositions include the step of providing the tumor vaccine of the present disclosure to one or more pharmaceutically acceptable carriers and, optionally, one or more adjuvants. Methods for making such formulations can be found in, for example, Remington's Pharmaceutical Sciences (Remington: The Science and Practice of Pharmacy, 19th ed., A. R. Gennaro (ed), Mack Publishing Co., N.J., 1995; R. Stribling et al., Proc. Natl. Acad. Sci. USA, 89:11277-11281 (1992); T. W. Kim et al., The Journal of Gene Medicine, 7(6): 749-758 (2005); S. F. Jia et al., Clinical Cancer Research, 9:3462 (2003); A. Shahiwala et al., Recent patents on drug delivery and formulation, 1:1-9 (2007); A. Barnes et al., Current Opinion in Molecular Therapeutics 2000 2:87-93 (2000), which references are incorporated herein by reference in their entirety).

In certain embodiments, the pharmaceutical composition containing the nucleic acids, expression vectors and/or host cells of the present disclosure is administered to a subject in need of treatment through local delivery into the target tissue or organ at the tumor site. In certain embodiments, the pharmaceutical composition is directly injected to a tumor site palpable through the skin using a syringe. In certain embodiments, the pharmaceutical composition is injected using an implantable dosing device connected to a catheter line or other medical access device, and may be used in conjunction with an imaging system guiding to the tumor site. In certain embodiments, an effective dose is directly injected to a tumor site visible in an exposed surgical field. In certain embodiments, the pharmaceutical composition (e.g. vector-coated gold particles) is bombarded to the tumor site using a gene gun which shots the particles directly into the tumor (see, for example, R. Muangmoonchai et al., Molecular Biology, 20(2):145-151 (2002)). In certain embodiments, the pharmaceutical compositions are administered to a subject through intravenous injection. In certain embodiments, the pharmaceutical compositions are administered orally or transmucosally to a subject. For references of methods of introducing therapeutic nucleic acids into cells or animals, see, for example, Yang, N-S., Crit. Rev. Biotechnol. 12:335-356 (1992); Anderson, W. F., Science 256:808-813 (1992); Miller, A. S., Nature 357:455-460 (1992); Crystal, R. G., Amer. J. Med. 92 (suppl 6A):44S-52S (1992); Zwiebel, J. A. et al., Ann N.Y. Acad. Sci. 618:394-404 (1991); McLachlin, J. R. et al., Prog. Nucl. Acid Res. Molec. Biol. 38:91-135 (1990); Kohn, D. B. et al., Cancer Invest. 7:179-192 (1989). These references are incorporated herein by reference in their entirety.

In certain embodiments, the pharmaceutical composition is administered at a therapeutically effective dosage. The term “therapeutic effective dosage” as used herein refers to an amount of the tumor vaccine, which, after administration, is effective to achieve the desired therapeutic result. A therapeutically effective amount of the tumor vaccine can vary from patient to patient according to factors such as the disease state, age, sex, and weight of the individual, and the ability of the tumor vaccine to elicit a desired response in the individual. The therapeutic effective dosage may be determined by starting with a low but safe dose and escalating to higher doses, while monitoring for therapeutic effects (e.g. a reduction in cancer cell growth) along with the presence of any deleterious side effects.

### EXAMPLES

The following Examples are set forth to aid in the understanding of the present disclosure, and should not be construed to limit in any way the scope of the invention as defined in the claims which follow thereafter.

### Example 1

### Construction of Adenovirus Vector Ad-HSP-hGM-CSF Vector

The adenovirus vector Ad-HSP-hGM-CSF is constructed as described below.

First, plasmid pIRES-E1a-blunt is constructed by replacing the EGFP fragment in plasmid pEGFP with an IRES fragment and an E1a fragment. IRES fragment is obtained by PCR from pIRES plasmid (Clontech, Mountain view, Calif., catalog #: 631605) using the pair of primers: CGGGATCCGCCCCTCTCCCTCCCCCCCCCCTAACGTTAC (forward primer), and GTGGCCATATTATCATCGTGTTTTTCAAAG (reverse primer). The PCR product is digested using BamHI. E1a fragment is obtained by PCR from pXC1 plasmid (Microbix Biosystems Inc., Ontario, Canada, product #: PD-01-03; see also McKinnon (1982) Gene 19:33-42) using the pair of primers: CCGACCGGTGACTGAAAATGAGACATATTATC (forward primer) and CGCTGTACAACCACACACGCAATCACAGG (reverse primer). The PCR product is digested using AgeI and BsrGI. Plasmid pEGFP (Clontech, Mountain view, Calif., catalog #: 6077-1) is digested with BamHI and BsrGI, and the resulting 2.7 kb fragment is purified and ligated with the obtained IRES fragment and E1a fragment. The E1a sequence is located upstream of the SV40 sequence in the vector. The resulting ligation product is confirmed by restriction digestion and named plasmid pIRES-E1a-blunt.

Next, plasmid pE2F-hGM-CSF-IRES-E1a is constructed by inserting a fragment containing both E2F promoter and human GM-CSF (hGM-CSF) gene to plasmid pIRES-E1a-blunt. pIRES-E1a-blunt is linearized with restriction enzymes Xho I and EcoRI. The 5.6 kb linearized product (referred to as Fragment A below) is analyzed using 0.5% agarose gel electrophoresis, and purified by gel extraction. Plasmid pORF9-hGM-CSF (Invivogen, San Diego, Calif., catalog #: porf-hgmcsf) is digested with Xho I and EcoRI, which digestion results in a 1.3 kb fragment and a 2.4 kb fragment. The 1.3 kb fragment (referred to as Fragment B below) which contains the E2F promoter and the hGM-CSF gene is purified and then ligated with Fragment A. The ligated product is confirmed by restriction digestion, and named plasmid pE2F-hGM-CSF-IRES-E1a. The hGM-CSF in the pE2F-hGM-CSF-IRES-E1a has a nucleotide sequence of SEQ ID NO: 28. The other 2.4 kb digestion fragment (referred to as Fragment C below) from plasmid pORF9-hGM-CSF is also purified by gel extraction for later use.

Plasmid pY1-HSP-amp is constructed by inserting CMV promoter and human Heat shock protein 70 (hHSP70) gene into plasmid pSL1190 (Pharmacia, catalog No. 27-4386; see also, Brosius J, DNA, 8(10): 759-77 (1989)). hHSP70 gene is obtained by PCR from human HSP70 cDNA clone (Origene, catalog #SC 116766) using the pair of primers: CCCAAGCTTATGGCCAAAGCCGCGGC (forward primer) and CGGGATCCACTAGTCTAATCTACCTC (reverse primer). The hHSP70 gene has a nucleotide sequence of SEQ ID NO: 27. The PCR product is digested with HindIII and BamHI. CMV promoter is obtained by PCR from pcDNA3.1 (Invitrogen, Carlsbad, Calif., catalog #: V860-20) using the pair of primers: GGAATTCCATATGCCAAGTACGCCCCCTATTG (forward primer) and CCCAAGCTTGTGAGTCGTATTAATTTC (reverse primer). The PCR product is digested using NdeI and HindIII. Plasmid pSL1190 is linearized with Nde I and BamHI, and the 3.2 kb linearized product is purified by gel extraction, and then ligated with the hHSP70 fragment and the CMV promoter fragment. The final ligation product is confirmed by restriction digestion, and named as plasmid pY1-HSP-amp.

Next, plasmid pORF9-hHSP70 is constructed by ligating Fragment C with a fragment from pY1-HSP-amp which contains CMV promoter and hHSP70 gene. Plasmid Y1-HSP-amp is digested with Xho I and EcoR I. The resulting 2.5 kb fragment which contains CMV promoter and hHSP70 gene is purified by gel extraction, and then ligated with Fragment C. The obtained ligation product is confirmed by restriction digestion and named as pORF9-hHSP.

Next, plasmid p-CMV-hHSP70-E2F-hGM-CSF-IRES-E1a is constructed by inserting a fragment containing CMV promoter, hHSP70 gene and SV40 pA into plasmid p-E2F-hGM-CSF-IRES-E1a. Plasmid p-E2F-hGM-CSF-IRES-E1a is linearized with Xho I and Pvu I, and the linearized 6.7 kb product (referred to as Fragment D below) is purified by gel extraction. Plasmid pORF9-hHSP is digested using Xho I and Pac I, and the resulting 3.0 kb fragment (referred to as Fragment E below) which contains CMV promoter, hHSP70 and SV40 pA is purified by gel extraction. Fragment D and Fragment E are ligated by DNA ligase. The ligated product is confirmed by restriction digestion and named pCMV-hHSP70-E2F-hGM-CSF-IRES-E1a.

Next, pShuttle-CMV-hHSP70-E2F-hGM-CSF-IRES-E1a is constructed by inserting the fragment of CMV-hHSP70-E2F-hGM-CSF-IRES-E1a from pCMV-hHSP70-E2F-hGM-CSF-IRES-E1a into pShuttle vector. p-CMV-hHSP70-E2F-hGM-CSF-IRES-E1a is digested with Xho I and Ssp I, and the resulting 6.0 kb fragment (referred to as Fragment F) which contains CMV promoter, hHSP70, SV40 pA, E2F promoter, hGM-CSF, IRES and E1a and SV40 pA, is purified by gel extraction. Shuttle plasmid pShuttle (Clontech, Mountain View, Calif., catalog #: K1650-1) is linearized using EcoR V and Sal I, and the resulting 6.5 kb linearized product (referred to as Fragment G below) is purified by gel extraction. Fragment F and Fragment G are ligated by DNA ligase. The ligated product is confirmed by restriction digestion, and named as pShuttle-CMV-hHSP70-E2F-hGM-CSF-IRES-E1a.

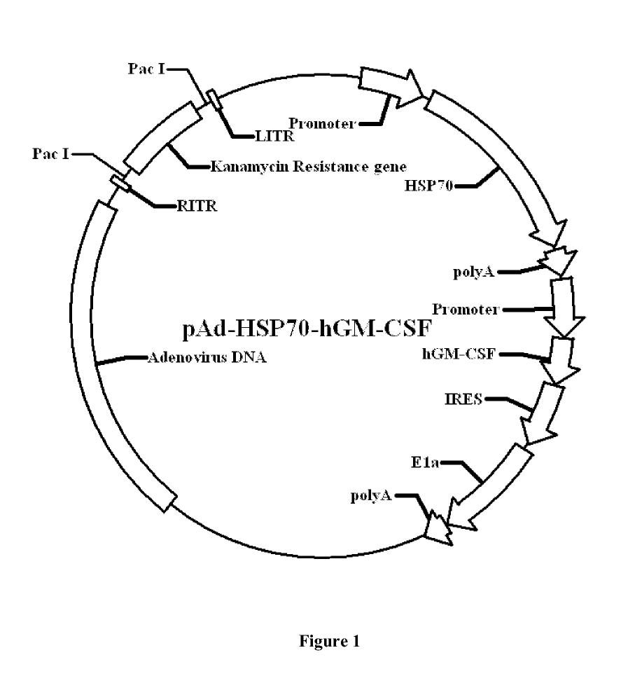

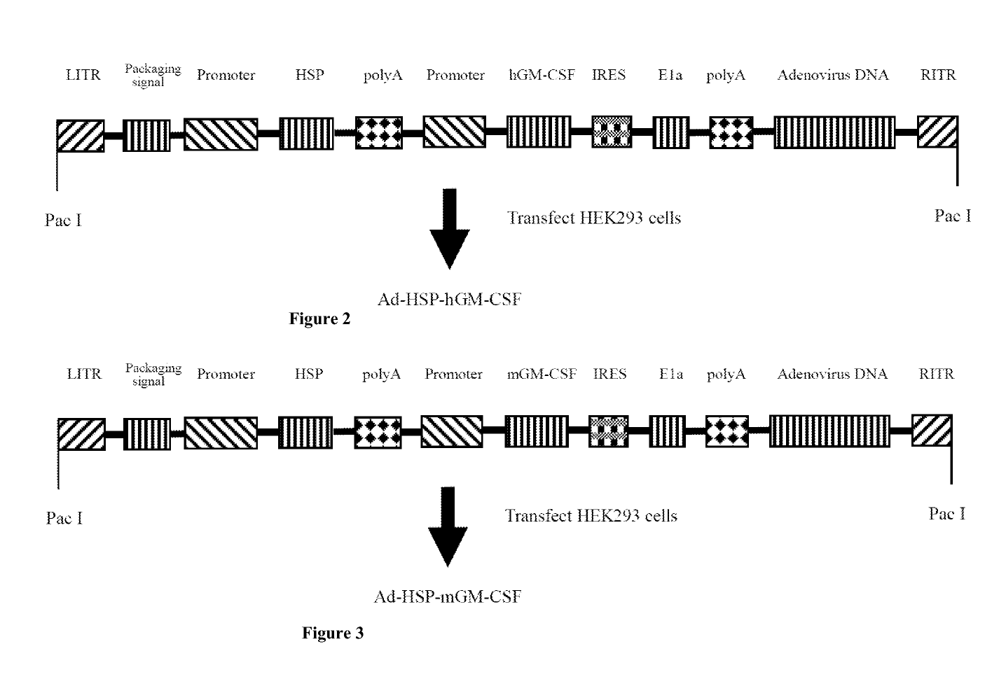

Finally, adenovirus vector Ad-HSP-hGM-CSF is made by homologous recombination of plasmid pShuttle-CMV-hHSP70-E2F-hGM-CSF-IRES-E1a (referred to as pShuttle-HSP-hGM-CSF) and pAdEasy-1 viral DNA plasmid (Stratagene, La Jolla, Calif., Catalog #240009). pShuttle-HSP-hGM-CSF, which contains a left arm homology sequence and a right arm homology sequence, is linearized with Pme I to yield the linearized product with the two homology sequences separately located at the two respective ends. The linearized product is cotransformed into E. coli strain BJ5183 with pAdEasy-1 viral DNA plasmid, and homologous recombination between pShuttle-HSP-hGM-CSF and pAdEASY takes place between the left and right homology sequences. Transformants are selected for kanamycin resistance, and the resulting recombinant vectors, which is referred to as pAd-HSP-hGM-CSF, are subsequently isolated and identified by restriction digestion. The identified pAd-HSP-hGM-CSF vector is sequenced to make sure that no mutation occurs in the inserted target genes. The schematic structure of the pAd-HSP-hGM-CSF is shown in FIG. 1. The pAd-HSP-hGM-CSF vector is digested with Pac I to provide a digestion product containing the Adenoviral DNA and the inserted genes, with the inverted terminal repeats (ITR) exposed at both ends. The digestion product is transfected into HEK293 cells to make the adenovirus-producing 293 cells, from which adenovirus Ad-HSP-hGM-CSF is recovered. The schematic structure of the genome of the adenovirus Ad-HSP-hGM-CSF is shown in FIG. 2.

### Example 2

### Construction of Adenovirus Vector Ad-HSP-mGM-CSF

The adenovirus vector Ad-HSP-mGM-CSF is constructed as described below.

First, plasmid pIRES-E1a-blunt is constructed by replacing the EGFP fragment in plasmid pEGFP with an IRES fragment and an E1a fragment. IRES fragment is obtained by PCR from pIRES plasmid (Clontech, Mountain view, Calif., catalog #: 631605) using the pair of primers: CGGGATCCGCCCCTCTCCCTCCCCCCCCCCTAACGTTAC (forward primer), and GTGGCCATATTATCATCGTGTTTTTCAAAG (reverse primer). The PCR product is digested using BamHI. E1a fragment is obtained by PCR from pXC1 plasmid (Microbix Biosystems Inc., Ontario, Canada, product #: PD-01-03; see also McKinnon (1982) Gene 19:33-42) using the pair of primers: CCGACCGGTGACTGAAAATGAGACATATTATC (forward primer) and CGCTGTACAACCACACACGCAATCACAGG (reverse primer). The PCR product is digested using AgeI and BsrGI. Plasmid pEGFP (Clontech, Mountain view, Calif., catalog #: 6077-1) is digested with BamHI and BsrGI, and the resulting 2.7 kb fragment is purified and ligated with the previously obtained IRES fragment and the E1a fragment. The E1a sequence is located upstream in the SV40 sequence of the vector. The resulting ligation product is confirmed by restriction digestion and named plasmid pIRES-E1a-blunt.

Next, plasmid pORF9-mGM-CSF-com is constructed by replacing the hGM-CSF gene in the plasmid pORF9-hGM-CSF with the mouse GM-CSF gene (mGM-CSF) from the plasmid pORF9-mGM-CSF. Plasmid pORF-hGM-CSF (available from Invivogen, San Diego, Calif., catalog #: porf-hgmcsf) is digested with SgrA I and Nhe I, and the resulting 3.2 kb fragment (referred to as Fragment H) is purified by gel extraction. Plasmid pORF-mGM-CSF (available from Invivogen, San Diego, Calif., catalog #: porf-mgmcsf) is digested with Age I and Nhe I, and the resulting 452 bp fragment (referred to as Fragment I) which contains mGM-CSF gene, is purified by gel extraction. Fragment H and Fragment I are ligated by DNA ligase. The ligated product is confirmed by restriction digestion and named plasmid pORF-mGM-CSF-com.

Next, plasmid pE2F-mGM-CSF-IRES-E1a is constructed by inserting a fragment containing both E2F promoter and mGM-CSF gene to plasmid pIRES-E1a-blunt. pIRES-E1a-blunt is linearized with restriction enzymes Xho I and EcoRI. The 5.6 kb linearized product (referred to as Fragment A′ below) is analyzed using 0.5% agarose gel electrophoresis, and purified by gel extraction. Plasmid pORF9-mGM-CSF-com is digested with Xho I and EcoRI, which digestion results in a 1.3 kb fragment and a 2.4 kb fragment. The 1.3 kb fragment (referred to as Fragment B′ below) which contains the E2F promoter and the mGM-CSF gene is purified and then ligated with Fragment A′. The ligated product is confirmed by restriction digestion, and is named plasmid pE2F-mGM-CSF-IRES-E1a. The mGM-CSF in the pE2F-mGM-CSF-IRES-E1a has a nucleotide sequence of SEQ ID NO: 31. The other 2.4 kb digestion fragment (referred to as Fragment C′ below) from plasmid pORF9-hGM-CSF is also purified by gel extraction for later use.

Plasmid pY1-HSP-amp is constructed by inserting CMV promoter and human Heat shock protein 70 (hHSP70) gene into plasmid pSL1190 (Pharmacia, catalog No. 27-4386; see also, Brosius J, DNA, 8(10): 759-77 (1989)). hHSP70 gene is obtained by PCR from human HSP70 cDNA clone (Origene, catalog #SC116766) using the pair of primers: CCCAAGCTTATGGCCAAAGCCGCGGC (forward primer) and CGGGATCCACTAGTCTAATCTACCTC (reverse primer). The hHSP70 gene has a nucleotide sequence of SEQ ID NO: 27. The PCR product is digested with HindIII and BamHI. CMV promoter is obtained by PCR from pcDNA3.1 (Invitrogen, Carlsbad, Calif., catalog #: V860-20) using the pair of primers: GGAATTCCATATGCCAAGTACGCCCCCTATTG (forward primer) and CCCAAGCTTGTGAGTCGTATTAATTTC (reverse primer). The PCR product is digested using NdeI and HindIII. Plasmid pSL1190 is linearized with Nde I and BamHI, and the 3.2 kb linearized product is purified by gel extraction, and then ligated with the hHSP70 fragment and the CMV promoter fragment. The final ligation product is confirmed by restriction digestion, and named as plasmid pY1-HSP-amp.

Next, plasmid pORF9-hHSP70 is constructed by ligating Fragment C′ with a fragment from pY1-HSP-amp which contains CMV promoter and hHSP70 gene. Plasmid Y1-HSP-amp is digested with Xho I and EcoR I. The resulting 2.5 kb fragment which contains CMV promoter and hHSP70 gene is purified by gel extraction, and is then ligated with Fragment C′. The obtained ligation product is confirmed by restriction digestion and named as pORF9-hHSP.

Next, plasmid p-CMV-hHSP70-E2F-mGM-CSF-IRES-E1a is constructed by inserting a fragment containing CMV promoter, hHSP70 gene and SV40 pA into plasmid p-E2F-mGM-CSF-IRES-E1a. Plasmid pE2F-mGM-CSF-IRES-E1a is linearized with Xho I and Pvu I, and the linearized 6.7 kb product (referred to as Fragment D′ below) is purified by gel extraction. Plasmid pORF9—HSP is digested using Xho I and Pac I, and the resulting 3.0 kb fragment (referred to as Fragment E′ below) which contains CMV promoter, hHSP70 and SV40 pA is purified by gel extraction. Fragment D′ and Fragment E′ are ligated by DNA ligase. The ligated product is confirmed by restriction digestion and named pCMV-hHSP70-E2F-mGM-CSF-IRES-E1a.

Next, pShuttle-CMV-hHSP70-E2F-mGM-CSF-IRES-E1a is constructed by inserting the fragment of CMV-hHSP70-E2F-mGM-CSF-IRES-E1a from pCMV-hHSP70-E2F-hGM-CSF-IRES-E1a into pShuttle vector. p-CMV-hHSP70-E2F-mGM-CSF-IRES-E1a is digested with Xho I and Ssp I, and the resulting 6.0 kb fragment (referred to as Fragment F′) which contains CMV promoter, hHSP70, SV40 pA, E2F promoter, mGM-CSF, IRES and E1a and SV40 pA, is purified by gel extraction. Shuttle plasmid pShuttle (Clontech, Mountain View, Calif., catalog #: K1650-1) is linearized using EcoR V and Sal I, and the resulting 6.5 kb linearized product (referred to as Fragment G′ below) is purified by gel extraction. Fragment F′ and Fragment G′ are ligated by DNA ligase. The ligated product is confirmed by restriction digestion, and named as pShuttle-CMV-hHSP70-E2F-mGM-CSF-IRES-E1a.

Finally, adenovirus vector Ad-HSP-mGM-CSF is made by homologous recombination of plasmid pShuttle-CMV-hHSP70-E2F-mGM-CSF-IRES-E1a (referred to as pShuttle-HSP-mGM-CSF) and pAdEasy-1 viral DNA plasmid (Stratagene, La Jolla, Calif., Catalog #240009). pShuttle-HSP-mGM-CSF, which contains a left arm homology sequence and a right arm homology sequence, is linearized with Pme I to yield the linearized product with the two homology sequences separated at the two respective ends. The linearize product is cotransformed into E. coli strain BJ5183 with pAdEasy-1 viral DNA plasmid, and homologous recombination between pShuttle-HSP-mGM-CSF and pAdEASY takes place between the left and right homology sequences. Transformants are selected for kanamycin resistance, and the resulting recombinant vector, which is referred to as pAd-HSP-mGM-CSF, is subsequently isolated and identified by restriction digestion. The identified pAd-HSP-mGM-CSF vector is sequenced to make sure that no mutation occurs in the inserted target genes. The pAd-HSP-mGM-CSF vector is digested with Pac I to provide a digestion product containing the Adenoviral DNA and the inserted genes, with the inverted terminal repeats (ITR) exposed at both ends. The digestion product is transfected into HEK293 cells to make the adenovirus-producing 293 cells, from which adenovirus Ad-HSP-mGM-CSF is recovered. The schematic structure of the genome of the adenovirus Ad-HSP-mGM-CSF is shown in FIG. 3.

### Example 3

### Expression of GM-CSF and HSP70 Proteins in Adenovirus Infected Cells

The adenovirus vector Ad-HSP-mGM-CSF is used to infect human cell lines including Hela and HepB cells, respectively, and murine B16 cells.

The adenovirus vector Ad-HSP-mGM-CSF is tested on each type of cell at a MOI of 10 (virus titer 10⁷), 100 (virus titer 10⁸), or 1000 (virus titer 10⁹). For each MOI, cells are incubated with the adenovirus vector for 12 hours, 24 hours, 36 hours, and 48 hours, respectively. At each time point, three wells of cells are collected for each type of cells. Therefore, there are 36 wells of testing samples for each type of cells. For each type of cells, 3 additional wells of cells are included as uninfected control, and the uninfected controls are collected after 12 hours of incubation. MOI is the ratio of the pfu of the virus deposited in a well divided by the number of the cells in the well. The number of the cells is calculated or measured using hemocytometer measurement method. The virus replication is confirmed by the virus titration assay.

The experiment is carried out as described below. Cells are cultured in a culture plate. When the cells reach 90% confluency, the cells are washed with phosphate buffer saline (PBS), and then trypsinized with 3 ml digestion solution for 2-3 minutes. When the adherent cells are detached, 10 ml complete Dulbecco's Modified Eagle Medium (DMEM) culture medium is added to the plate to suspend the cells. The number of cells in the cell suspension is counted and the cell suspension is diluted by complete DMEM to make a final concentration of 10⁶ cells/ml. The cell suspension of each type of cells is then added to 12-well plates in the amount of 1 ml/well.

The cells are cultured until they reach enough confluency. The culture medium is discarded, and the virus solution diluted with complete DMEM is added at 1 ml/well to the cell plates at a MOI of 10, 100, or 1000. At the 12^{th} hour, 24^{th} hour, 36^{th} hour and 48^{th} hour, three wells of cells are collected for each MOI and each cell type. The cell culture is collected into a 1.5 ml centrifuge tube for later determination of the protein expression in the supernatant. Then the cells remaining in the wells are washed gently with PBS for 3 times, and then trypsinized and collected to a separate centrifuge tube. The collected cells are centrifuged at 2500 rpm for 5 minutes, and then stored in −80° C. refrigerator after discarding the supernatants.

After the 48-hour experimental period, when all samples are collected and prepared, protein extraction is subsequently performed. The 5× Extraction Reagent is diluted to 1× using cold (4° C.) deionized water, and Protease Inhibitor Cocktail Tablets are added (1 tablet/10 ml extraction solution). The extraction solution is added to the collected cells at a ratio of 10⁶-10⁷ cells:1 ml protein extraction solution, and the cells are resuspended to a homogeneous solution without any significant cell pellet. The resuspended cells are placed on ice for 30 minutes with intermittent shakings or short ultrasonic treatment. The cells are then centrifuged at 21000 g, 4° C. for 10 minutes, and the supernatants are transferred to new centrifuge tubes, which contain the total proteins.

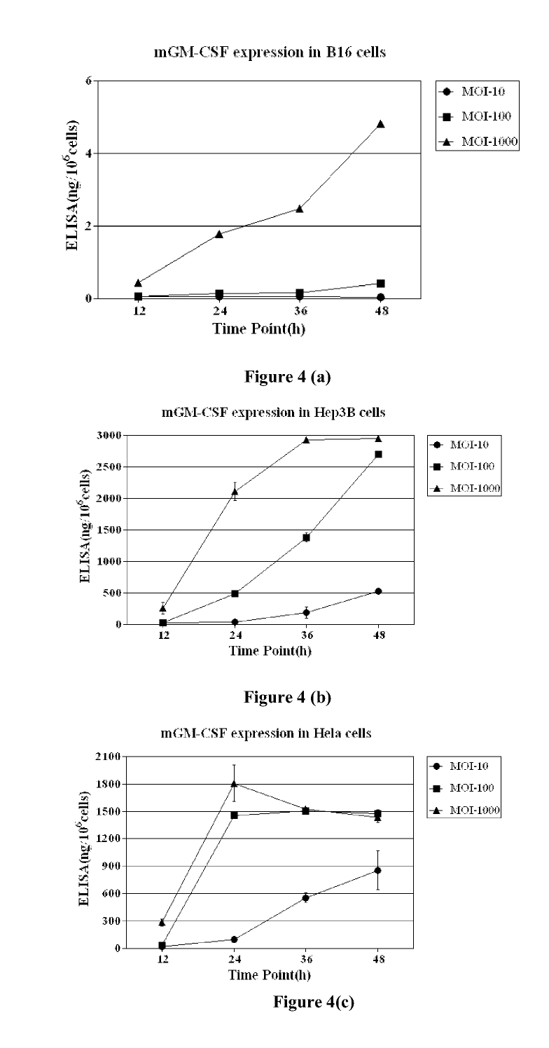

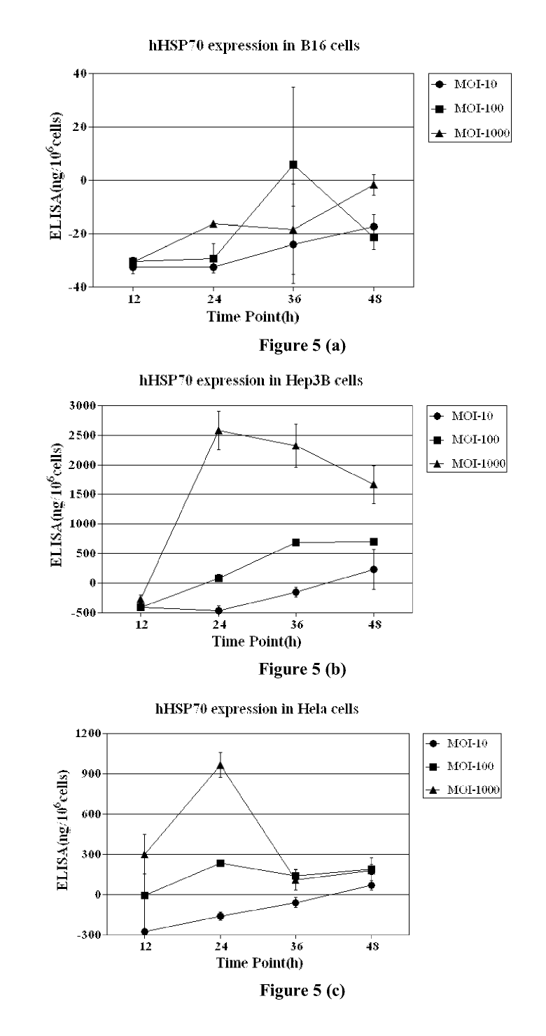

The expression level of mGM-CSF and hHSP70 are determined using ELISA kits. The expression of mGM-CSF is determined using Quantikine® Mouse GM-CSF Immunoassay, purchased from R&D Systems (Catalog #: MGM00). The expression of hHSP70 is determined using StressXpress® Hsp70 ELISA Kit, purchased from Assay Designs (Catalog #: EKS-700B). The concentrations of the mGM-CSF and hHSP70 are measured following the instruction of the ELISA Kit. The results are shown in FIGS. 4(a), 4(b), and 4(c), and FIGS. 5(a), 5(b), and 5(c).

mGM-CSF expression levels are shown in FIG. 4. mGM-CSF is expressed in all of the three cell types, in which Hep3B cells express the highest amount of mGM-CSF and B 16 cells express the lowest amount. In all three cell types, the expression levels increase with the incubation time, of which the expression levels of Hep3B cells reach the highest amount at about 48 hour. At the same time point, the expression amount of mGM-CSF is generally higher as the MOI increases from 10 to 1000. At each time point, the protein expression levels in the supernatants are higher than in the cell pellet, suggesting the proteins are secreted.

hHSP70 expression levels are shown in FIG. 5. hHSP70 is expressed in all of the three cell types, in which Hep3B cells express the highest amount of hHSP70 and B 16 cells express the lowest amount. In B16 cells, no expression is detected except for samples collected at 36 hours at MOI 100. This may be due to the low infectivity of human adenovirus to murine cells. In all three cell types, the expression levels increase with the incubation time, of which the expression levels of Hep3B cells reach the highest amount at about 24 hour. At the same time point, the expression amount of hHSP70 is generally higher as the MOI increases from 10 to 1000. In Hep3B cells, the expression levels in the cell pellets are higher than in the supernatant. In Hela cells, the expression levels in the supernatants are higher than in the cell pellet.

### Example 4

### Amplification of the Adenovirus Vector in Cells

The virus vector is amplified in HEK293 cells in T flask and purified by CsCl gradient centrifugation. The virus yield is determined by virus titration assay and spectrophotometer method. The virus is stored at −70° C. for later use.

### Example 5

### Comparison of Cytotoxicity of Ad-HSP-hGM-CSF with Other Virus Vectors

The cytotoxicity of the adenovirus vector Ad-HSP-hGM-CSF is compared with other virus vectors in different tumor cell lines, including A549 (adenocarcinomic human alveolar basal epithelial cells, purchased from Fudan University, Shanghai, China), Hepal-6 (mouse liver cancer cells, purchased from China Centre for Type Culture Collection (CCTCC), Wuhan, China), MCF-7 (human breast cancer cells, purchased from Fudan University, Shanghai, China), TCa8113 (human tongue carcinoma cells, purchased from Chinese Academy of Science, Shanghai), ACC-M (adenoid cystic carcinoma cell clones highly metastatic to the lung, purchased from CCTCC, Wuhan, China), HeLa (human cervical cancer cells, purchased from Fudan University, Shanghai, China), Hep3B (human liver cancer cells, purchased from Fudan University, Shanghai, China), and Lncap (androgen-sensitive human prostate adenocarcinoma cells, purchased from Chinese Academy of Science, Shanghai). Ad-GM-CSF (constructed by homologus recombination between plasmid pORF9-hGM-CSF and pAdEasy-1 viral DNA plasmid), ONYX015 (a E1B-55k deleted adenovirus that selectively replicates in p53-deficient cancer cells) (gift from Chiba University), and Ad-CMV-HSP (gift from Shanghai Sunway Biotech) are tested in parallel for comparison of the cytotoxicity effects.

Cells are grown in a 75 cm² cell culture bottle until 90% confluent. Culture medium is discarded, and 2 ml trypsin solution is added to trypsinize the cells. When cells are detached, 10 ml culture medium containing 2% fetal bovine serum is added to suspend the cells. The number of cells in the cell suspension is counted and the cell suspension is diluted to a final concentration of 2×10⁵ cells/ml by the culture medium containing 2% fetal bovine serum. The amount of the cells are calculated or measured using hemocytometer measurement method. The cell suspension is then added to 96-well plates at 50 μl/well. The 96-well plates are incubated for 1 day in a CO₂ incubator.

The virus solution is diluted with the culture medium containing 2% fetal bovine serum, and is added at 50 μl/well to the cell plates in a MOI of 300, 100 and 30. For each MOI, the infection is performed in triplicate. The blank control contains 100 μl culture medium/well. The 96-well plates are incubated for 6 days in a CO₂ incubator.

The cytotoxicity for each vector in each cell type is determined using the MTS detection kit: CellTiter 96 Aqueous One Solution Cell Proliferation Assay (MTS), purchased from Promega (Catalog #G3580). Briefly, each type of cells are inoculated to 96-well plates and cultured at 37° C. until 95% confluent. Viruses are added to the cells at the corresponding MOIs and the cultures continue to grow at 37° C. CellTiter 96® AQueous One Solution Reagent is added to each culture at a v/v ratio of 1:5. After further incubation at 37° C. for 1-4 hours, the plates are measured for absorbance at 490 nm using a spectrophotometer. The cell viability for each cell type is calculated by dividing the optical density measured for cells after virus infection by the optical density measured for control cells without virus infection.

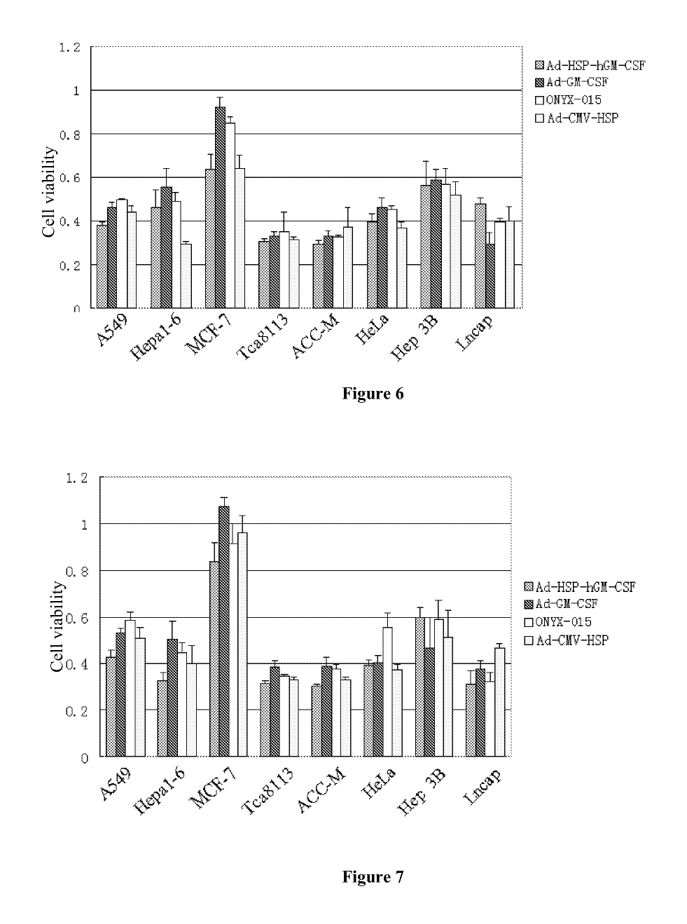

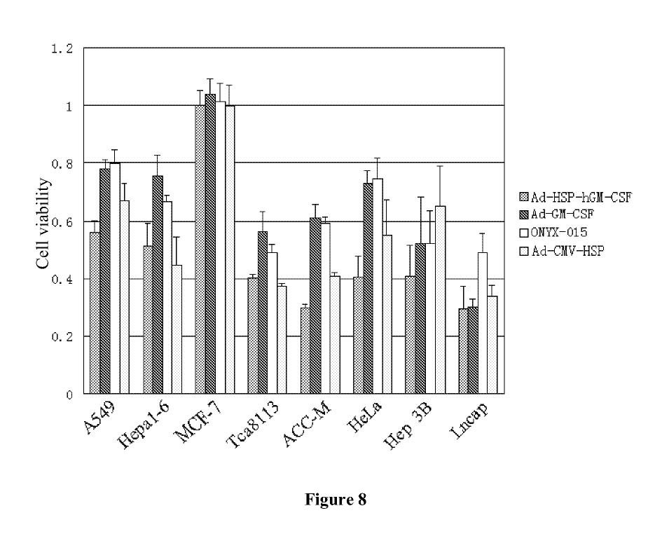

The results are shown in FIGS. 6-8. Ad-HSP-hGM-CSF show significant activity in inhibiting tumor cell growth in all the cell types tested and at all the MOIs tested. TCa8113, ACC-M, HeLa, Hep3B, and Lncap cells are killed, by more than 50% after treatment with Ad-HSP-hGM-CSF while the other cells are killed by less than 50% after the treatment. The anti-tumor activity of Ad-HSP-hGM-CSF is comparable to, and in many cases better than those seen with the vector Ad-GM-CSF, ONYX015, and Ad-CMV-HSP.

### Example 6

### Comparison of Cell Toxicity of Ad-HSP-mGM-CSF on Different Tumor Cells

The cytotoxicity of the recombinant oncolytic adenovirus Ad-HSP-mGM-CSF is tested in different human tumor cell lines including: HEK293, Hep3B, A549, 7402 (hepatocellular carcinoma cell line, purchased from liver cancer institute, Fudan University, Shanghai, China), 7721 (hepatocellular carcinoma cell line, purchased from liver cancer institute, Fudan University, Shanghai, China) and Hela, all of which are maintained in RPMI 1640 complete medium. A normal cell line (2BS) is used as control. HEK 293 cell line is used as a positive control, as this cell line has been transformed with E1 gene of adenovirus and thus can support the growth of any type of adenovirus. The specific tumor killing effects are compared.

Each cell line is divided into 5 groups, infected with 0.1, 1, 10, 100 and 1000 MOIs of the Ad-HSP-mGM-CSF, respectively.

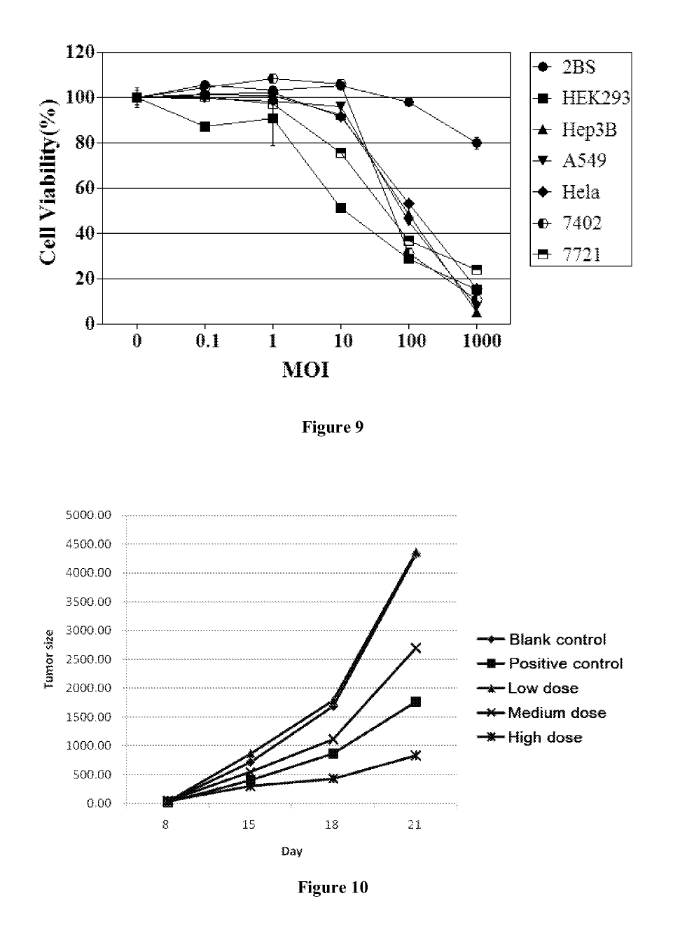

The experiment is performed in the same way as described in Example 5. The cytotoxicity is determined using MTS detection kit as described in Example 5. Cell viability is calculated by dividing the optical density measured for cells treated with virus infection by that measured for control cells without virus infection. As shown in FIG. 9, the cytotoxicity of the adenovirus vector is different between the normal cells and the tumor cells. For the 2BS cells, the cell viability is above 80% when infected with a MOI of 1000 of virus. However, the viability of tumor cell lines declines as the MOI of the virus infection increases. For A549 cells and 7402 cells, the virus begins to show significant cytotoxicity at the MOI of 100, and the cell viability drops rapidly with further increase of MOI. For Hep3B cells, 7721 cells and Hela cells, as well as the positive control HEK 293 cells, the virus begins to show significant cytotoxicity at the MOI of 10, and the cell viability drops rapidly with further increase of MOI.

### Example 7

### Tumor Reduction in Mice

The therapeutic effects of Ad-HSP-mGM-CSF are studied in a C57 mice B16 tumor model. Three experimental groups are set, including a high dose group, medium dose group and low dose group. A blank control group (10% glycerin PBS) and a positive control group (2 mg/kg Cisplatin) are also included. The dosage groups are shown in Table 3.

TABLE 3

| Tumor | | Dosage (Virus |
|---|---|---|
| model | Group | Particle/ml) |
| B16 | High dose | 2.42 × 10¹⁰ |
| Tumor | Medium dose | 2.42 × 10⁹ |
| model | Low dose | 2.42 × 10⁸ |
| | Positive control | Cisplatin 2 mg/kg |
| | Blank control | 10% glycerin PBS |

B16 cells are subcutaneously inoculated to C57 mice under the abdomen, at an inoculation dosage of 7×10⁵ cells each mice (in a total number of 3.85×10⁷ B16 cell). On the 8^{th} day after the inoculation, the tumor volume reaches a threshold size (50-70 mm³), and the animals are ready to be used in the study.

C57 mice bearing B16 tumor that have appropriate tumor size and shape, have no concurring tumors and are in good behavior, are selected. The mice are graded according to their tumor size and then randomly divided into groups. At the day before the initiation of drug administration, the tumor size of each mouse is determined in vivo by an external caliper measurement of the length and width of the subcutaneous tumor. The volume of drug administration for the first day is calculated as 25% of the tumor size, and the volume remain the same for the subsequent days. Cisplatin is administered in a volume of 0.01 ml/g, and the concentration is 0.2 mg/ml.

The mice receive the predetermined dosages of drugs, respectively, through intratumor injection of the Ad-HSP-mGM-CSF virus once a day for 5 consecutive days. The positive control is administered via the intraperitoneal route once a day for 5 consecutive days. 10% glycerin PBS is administered via intratumor injection to animals of the blank control group, once a day for 5 consecutive days. Every three days, the tumor size of each mouse is determined in vivo by an external caliper measurement of the length and width of the subcutaneous tumor, and the body weight is determined at the same interval as well. The mice are sacrificed when their tumor size exceeds 2000 mm³.

The tumor size is calculated for each experimental group at different time points using the following calculation equation:

$$Tumor Size = \frac{length {width}^{2}}{2}$$

The study results are shown in FIG. 10. The average tumor size in the low dose group is almost the same as the blank control, indicating that the low dose has little tumor inhibition effect. The average tumor size in the medium dose group is lower than the blank control but higher than the positive control, indicating that the medium dose has moderate tumor inhibition effects. The average tumor size in the high dose group is better than the positive group, showing that the high dose has significant inhibition on the tumor growth. The survival periods of the mice are also recorded, with the high dose group has the highest survival rate at the end of the study, and the low dose group has the lowest survival rate. The results show that the tumor inhibition effects of the virus are dose-dependent. The results also show that the survival curve is generally in consistency with the tumor size curve.

With respect to the use of substantially any plural and/or singular terms herein, those having skill in the art can translate from the plural to the singular and/or from the singular to the plural as is appropriate to the context and/or application.

In addition, where features or aspects of the disclosure are described in terms of Markush groups, those skilled in the art will recognize that the disclosure is also thereby described in terms of any individual member or subgroup of members of the Markush group.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A composition comprising:
a first polynucleotide encoding an antigen presenting polypeptide,
a second polynucleotide encoding a cytokine,
and at least one promoter,
wherein the at least on promoter is operably linked to at least one of the first polynucleotide and the second polynucleotide.

2. An expression vector comprising:
a first polynucleotide segment encoding an antigen presenting polypeptide,
a second polynucleotide segment encoding a cytokine,
and at least one promoter,
wherein the at least one promoter is operably linked to at least one of the first polynucleotide segment and the second polynucleotide segment.

3. The composition of claim 1, wherein the antigen presenting polypeptide is a Heat Shock Protein.

4. The composition of claim 1, wherein the Heat Shock Protein is selected from the group consisting of Hsp10, Hsp20, Hsp27, Hsp40, Hsp60, Hsp70, Hsp71, Hsp72, Hsp90, Hsp104, Hsp110, alpha-B-crystallin, and glucose-regulated protein.

5. The composition of claim 4, wherein the Heat Shock Protein is Hsp70.

6. The composition of claim 1, wherein the antigen presenting polypeptide has at least 70% sequence identity to Hsp70.

7. The composition of claim 1, wherein the cytokine is selected from the group consisting of macrophage-colony stimulating factors, granulocyte-colony stimulating factors, macrophage/granulocyte-colony stimulating factors, stem cell factors, and erythropoietin.

8. The composition of claim 1, further comprising a third polynucleotide encoding a cytokine operably linked to a promoter.

9. The composition of claim 1, wherein the first polynucleotide is operably linked to a constitutive promoter and the second polynucleotide is operably linked to a tumor specific promoter.

10. The composition of claim 1, wherein the at least one promoter is a eukaryotic promoter.

11. The composition of claim 1, wherein the at least one promoter is a tumor specific promoter.

12. The expression vector of claim 2, wherein the expression vector is selected from the group consisting of plasmid, virus, phage, and cosmid.

13. The expression vector of claim 12, wherein the expression vector is a virus vector.

14. The expression vector of claim 13, wherein the virus vector is a vector derived from one or more viruses selected from the group consisting of adenovirus, retrovirus, lentivirus, vaccinia virus, herpes simplex virus, vesicular stomatitis virus, measles virus, respiratory and enteric orphan virus, Newcastle disease virus, and Sendai virus.

15. The expression vector of claim 13, wherein the virus vector is a replication competent vector.

16. The expression vector of claim 15, wherein the virus vector is an adenovirus vector containing an E1a gene.

17. The expression vector of claim 16, wherein the E1a gene is operably linked to a tumor specific promoter.

18. An isolated host cell containing a composition of claim 1.

19. A pharmaceutical composition comprising a pharmaceutically acceptable carrier and the composition of claim 1.

20. A method of treating a cancer, comprising administering to a subject the pharmaceutical composition of claim 19.


SEQUENCE LISTING (31 sequences)

SEQ ID NO: 1 - DNA, 1926 bp, Homo sapiens
atggccaaagccgcggcgatcggcatcgacctgggcaccacctactcctgcgtgggggtgttccaacacggcaaggtggagatcatcgccaacgaccagggcaaccgcaccacccccagctacgtggccttcacggacaccgagcggctcatcggggatgcggccaagaaccaggtggcgctgaacccgcagaacaccgtgtttgacgcgaagcggctgattggccgcaagttcggcgacccggtggtgcagtcggacatgaagcactggcctttccaggtgatcaacgacggagacaagcccaaggtgcaggtgagctacaagggggagaccaaggcattctaccccgaggagatctcgtccatggtgctgaccaagatgaaggagatcgccgaggcgtacctgggctacccggtgaccaacgcggtgatcaccgtgccggcctacttcaacgactcgcagcgccaggccaccaaggatgcgggtgtgatcgcggggctcaacgtgctgcggatcatcaacgagcccacggccgccgccatcgcctacggcctggacagaacgggcaagggggagcgcaacgtgctcatctttgacctgggcgggggcaccttcgacgtgtccatcctgacgatcgacgacggcatcttcgaggtgaaggccacggccggggacacccacctgggtggggaggactttgacaacaggctggtgaaccacttcgtggaggagttcaagagaaaacacaagaaggacatcagccagaacaagcgagccgtgaggcggctgcgcaccgcctgcgagagggccaagaggaccctgtcgtccagcacccaggccagcctggagatcgactccctgtttgagggcatcgacttctacacgtccatcaccagggcgaggttcgaggagctgtgctccgacctgttccgaagcaccctggagcccgtggagaaggctctgcgcgacgccaagctggacaaggcccagattcacgacctggtcctggtcgggggctccacccgcatccccaaggtgcagaagctgctgcaggacttcttcaacgggcgcgacctgaacaagagcatcaaccccgacgaggctgtggcctacggggcggcggtgcaggcggccatcctgatgggggacaagtccgagaacgtgcaggacctgctgctgctggacgtggctcccctgtcgctggggctggagacggccggaggcgtgatgactgccctgatcaagcgcaactccaccatccccaccaagcagacgcagatcttcaccacctactccgacaaccaacccggggtgctgatccaggtgtacgagggcgagagggccatgacgaaagacaacaatctgttggggcgcttcgagctgagcggcatccctccggcccccaggggcgtgccccagatcgaggtgaccttcgacatcgatgccaacggcatcctgaacgtcacggccacggacaagagcaccggcaaggccaacaagatcaccatcaccaacgacaagggccgcctgagcaaggaggagatcgagcgcatggtgcaggaggcggagaagtacaaagcggaggacgaggtgcagcgcgagagggtgtcagccaagaacgccctggagtcctacgccttcaacatgaagagcgccgtggaggatgaggggctcaagggcaagatcagcgaggcggacaagaagaaggtgctggacaagtgtcaagaggtcatctcgtggctggacgccaacaccttggccgagaaggacgagtttgagcacaagaggaaggagctggagcaggtgtgtaaccccatcatcagcggactgtaccagggtgccggtggtcccgggcctgggggcttcggggctcagggtcccaagggagggtctgggtcaggccccaccattgaggaggtagattag

SEQ ID NO: 2 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 3 - DNA, 2523 bp, Homo sapiens
atgtcggtggtgggcatagacctgggcttccagagctgctacgtcgctgtggcccgcgccggcggcatcgagactatcgctaatgagtatagcgaccgctgcacgccggcttgcatttcttttggtcctaagaatcgttcaattggagcagcagctaaaagccaggtaatttctaatgcaaagaacacagtccaaggatttaaaagattccatggccgagcattctctgatccatttgtggaggcagaaaaatctaaccttgcatatgatattgtgcagttgcctacaggattaacaggtataaaggtgacatatatggaggaagagcgaaattttaccactgagcaagtgactgccatgcttttgtccaaactgaaggagacagccgaaagtgttcttaagaagcctgtagttgactgtgttgtttcggttccttgtttctatactgatgcagaaagacgatcagtgatggatgcaacacagattgctggtcttaattgcttgcgattaatgaatgaaaccactgcagttgctcttgcatatggaatctataagcaggatcttcctgccttagaagagaaaccaagaaatgtagtttttgtagacatgggccactctgcttatcaagtttctgtatgtgcatttaatagaggaaaactgaaagttctggccactgcatttgacacgacattgggaggtagaaaatttgatgaagtgttagtaaatcacttctgtgaagaatttgggaagaaatacaagctagacattaagtccaaaatccgtgcattattacgactctctcaggagtgtgagaaactcaagaaattgatgagtgcaaatgcttcagatctccctttgagcattgaatgttttatgaatgatgttgatgtatctggaactatgaatagaggcaaatttctggagatgtgcaatgatctcttagctagagtggagccaccacttcgtagtgttttggaacaaaccaagttaaagaaagaagatatttatgcagtggagatagttggtggtgctacacgaatccctgcggtaaaagagaagatcagcaaatttttcggtaaagaacttagtacaacattaaatgctgatgaagctgtcactcgaggctgtgcattgcagtgtgccatcttatcgcctgctttcaaagtcagagaattttctatcactgatgtagtaccatatccaatatctctgagatggaattctccagctgaagaagggtcaagtgactgtgaagtcttttccaaaaatcatgctgctcctttctctaaagttcttacattttatagaaaggaacctttcactcttgaggcctactacagctctcctcaggatttgccctatccagatcctgctatagctcagttttcagttcagaaagtcactcctcagtctgatggctccagttcaaaagtgaaagtcaaagttcgagtaaatgtccatggcattttcagtgtgtccagtgcatctttagtggaggttcacaagtctgaggaaaatgaggagccaatggaaacagatcagaatgcaaaggaggaagagaagatgcaagtggaccaggaggaaccacatgttgaagagcaacagcagcagacaccagcagaaaataaggcagagtctgaagaaatggagacctctcaagctggatccaaggataaaaagatggaccaaccaccccaagccaagaaggcaaaagtgaagaccagtactgtggacctgccaatcgagaatcagctattatggcagatagacagagagatgctcaacttgtacattgaaaatgagggtaagatgatcatgcaggataaactggagaaggagcggaatgatgctaagaacgcagtggaggaatatgtgtatgaaatgagagacaagcttagtggtgaatatgagaagtttgtgagtgaagatgatcgtaacagttttactttgaaactggaagatactgaaaattggttgtatgaggatggagaagaccagccaaagcaagtttatgttgataagttggctgaattaaaaaatctaggtcaacctattaagatacgtttccaggaatctgaagaacgaccaaaattatttgaagaactagggaaacagatccaacagtatatgaaaataatcagctctttcaaaaacaaggaggaccagtatgatcatttggatgctgctgacatgacaaaggtagaaaaaagcacaaatgaagcaatggagtggatgaataacaagctaaatctgcagaacaagcagagtttgaccatggatccagttgtcaagtcaaaagagattgaagctaaaattaaggagctgacaagtacttgtagccctataatttcaaagcccaaacccaaagtggaacctccaaaagaggaacaaaaaaatgcagagcagaatggaccagtggatggacaaggagacaacccaggcccccaggctgctgagcagggtacagacacagctgtgccttcggattcagacaagaagcttcctgaaatggacattgattga

SEQ ID NO: 4 - protein, 840 aa, Homo sapiens
MSVVGIDLGFQSCYVAVARAGGIETIANEYSDRCTPACISFGPKNRSIGAAAKSQVISNAKNTVQGFKRFHGRAFSDPFVEAEKSNLAYDIVQLPTGLTGIKVTYMEEERNFTTEQVTAMLLSKLKETAESVLKKPVVDCVVSVPCFYTDAERRSVMDATQIAGLNCLRLMNETTAVALAYGIYKQDLPALEEKPRNVVFVDMGHSAYQVSVCAFNRGKLKVLATAFDTTLGGRKFDEVLVNHFCEEFGKKYKLDIKSKIRALLRLSQECEKLKKLMSANASDLPLSIECFMNDVDVSGTMNRGKFLEMCNDLLARVEPPLRSVLEQTKLKKEDIYAVEIVGGATRIPAVKEKISKFFGKELSTTLNADEAVTRGCALQCAILSPAFKVREFSITDVVPYPISLRWNSPAEEGSSDCEVFSKNHAAPFSKVLTFYRKEPFTLEAYYSSPQDLPYPDPAIAQFSVQKVTPQSDGSSSKVKVKVRVNVHGIFSVSSASLVEVHKSEENEEPMETDQNAKEEEKMQVDQEEPHVEEQQQQTPAENKAESEEMETSQAGSKDKKMDQPPQAKKAKVKTSTVDLPIENQLLWQIDREMLNLYIENEGKMIMQDKLEKERNDAKNAVEEYVYEMRDKLSGEYEKFVSEDDRNSFTLKLEDTENWLYEDGEDQPKQVYVDKLAELKNLGQPIKIRFQESEERPKLFEELGKQIQQYMKIISSFKNKEDQYDHLDAADMTKVEKSTNEAMEWMNNKLNLQNKQSLTMDPVVKSKEIEAKIKELTSTCSPIISKPKPKVEPPKEEQKNAEQNGPVDGQGDNPGPQAAEQGTDTAVPSDSDKKLPEMDID

SEQ ID NO: 5 - DNA, 2565 bp, Homo sapiens
atgcccccgtgttcgggcggggacggctccacccctcctgggccctcccttcgggacagggactgtcccgcccagagtgctgaatacccgcgcgaccgtctggatccccgcccaggaagcccctctgaagcctcctcgccgccgtttctgagaagcagggcacctgttaactggtaccaagaaaaggcccaagtgtttctctggcatctgatggtgtctggatccaccactctactctgtctctggaaacagcccttccacgtctctgcattccctgtcaccgcgtcactggccttcagacagagccaaggtgcagggcaacacctctacaaggatctgcagccatttatattgcttaggctactgatgcctgaggaaacccagacccaagaccaaccgatggaggaggaggaggttgagacgttcgcctttcaggcagaaattgcccagttgatgtcattgatcatcaatactttctactcgaacaaagagatctttctgagagagctcatttcaaattcatcagatgcattggacaaaatccggtatgaaagcttgacagatcccagtaaattagactctgggaaagagctgcatattaaccttataccgaacaaacaagatcgaactctcactattgtggatactggaattggaatgaccaaggctgacttgatcaataaccttggtactatcgccaagtctgggaccaaagcgttcatggaagctttgcaggctggtgcagatatctctatgattggccagttcggtgttggtttttattctgcttatttggttgctgagaaagtaactgtgatcaccaaacataacgatgatgagcagtacgcttgggagtcctcagcagggggatcattcacagtgaggacagacacaggtgaacctatgggtcgtggaacaaaagttatcctacacctgaaagaagaccaaactgagtacttggaggaacgaagaataaaggagattgtgaagaaacattctcagtttattggatatcccattactctttttgtggagaaggaacgtgataaagaagtaagcgatgatgaggctgaagaaaaggaagacaaagaagaagaaaaagaaaaagaagagaaagagtcggaagacaaacctgaaattgaagatgttggttctgatgaggaagaagaaaagaaggatggtgacaagaagaagaagaagaagattaaggaaaagtacatcgatcaagaagagctcaacaaaacaaagcccatctggaccagaaatcccgacgatattactaatgaggagtacggagaattctataagagcttgaccaatgactgggaagatcacttggcagtgaagcatttttcagttgaaggacagttggaattcagagcccttctatttgtcccacgacgtgctccttttgatctgtttgaaaacagaaagaaaaagaacaacatcaaattgtatgtacgcagagttttcatcatggataactgtgaggagctaatccctgaatatctgaacttcattagaggggtggtagactcggaggatctccctctaaacatatcccgtgagatgttgcaacaaagcaaaattttgaaagttatcaggaagaatttggtcaaaaaatgcttagaactctttactgaactggcggaagataaagagaactacaagaaattctatgagcagttctctaaaaacataaagcttggaatacacgaagactctcaaaatcggaagaagctttcagagctgttaaggtactacacatctgcctctggtgatgagatggtttctctcaaggactactgcaccagaatgaaggagaaccagaaacatatctattatatcacaggtgagaccaaggaccaggtagctaactcagcctttgtggaacgtcttcggaaacatggcttagaagtgatctatatgattgagcccattgatgagtactgtgtccaacagctgaaggaatttgaggggaagactttagtgtcagtcaccaaagaaggcctggaacttccagaggatgaagaagagaaaaagaagcaggaagagaaaaaaacaaagtttgagaacctctgcaaaatcatgaaagacatattggagaaaaaagttgaaaaggtggttgtgtcaaaccgattggtgacatctccatgctgtattgtcacaagcacatatggctggacagcaaacatggagagaatcatgaaagctcaagccctaagagacaactcaacaatgggttacatggcagcaaagaaacacctggagataaaccctgaccattccattattgagaccttaaggcaaaaggcagaggctgataagaacgacaagtctgtgaaggatctggtcatcttgctttatgaaactgcgctcctgtcttctggcttcagtctggaagatccccagacacatgctaacaggatctacaggatgatcaaacttggtctgggtattgatgaagatgaccctactgctgatgataccagtgctgctgtaactgaagaaatgccaccccttgaaggagatgacgacacatcacgcatggaagaagtagactaa

SEQ ID NO: 6 - protein, 854 aa, Homo sapiens
MPPCSGGDGSTPPGPSLRDRDCPAQSAEYPRDRLDPRPGSPSEASSPPFLRSRAPVNWYQEKAQVFLWHLMVSGSTTLLCLWKQPFHVSAFPVTASLAFRQSQGAGQHLYKDLQPFILLRLLMPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 7 - DNA, 2040 bp, Homo sapiens
atgataagtgccagccgagctgcagcagcccgtctcgtgggcgccgcagcctcccggggccctacggccgcccgccaccaggatagctggaatggccttagtcatgaggcttttagacttgtttcaaggcgggattatgcatcagaagcaatcaagggagcagttgttggtattgatttgggtactaccaactcctgcgtggcagttatggaaggtaaacaagcaaaggtgctggagaatgccgaaggtgccagaaccaccccttcagttgtggcctttacagcagatggtgagcgacttgttggaatgccggccaagcgacaggctgtcaccaacccaaacaatacattttatgctaccaagcgtctcattggccggcgatatgatgatcctgaagtacagaaagacattaaaaatgttccctttaaaattgtccgtgcctccaatggtgatgcctgggttgaggctcatgggaaattgtattctccgagtcagattggagcatttgtgttgatgaagatgaaagagactgcagaaaattacttggggcacacagcaaaaaatgctgtgatcacagtcccagcttatttcaatgactcgcagagacaggccactaaagatgctggccagatatctggactgaatgtgcttcgggtgattaatgagcccacagctgctgctcttgcctatggtctagacaaatcagaagacaaagtcattgctgtatatgatttaggtggtggaacttttgatatttctatcctggaaattcagaaaggagtatttgaggtgaaatccacaaatggggataccttcttaggtggggaagactttgaccaggccttgctacggcacattgtgaaggagttcaagagagagacaggggttgatttgactaaagacaacatggcacttcagagggtacgggaagctgctgaaaaggctaaatgtgaactctcctcatctgtgcagactgacatcaatttgccctatcttacaatggattcttctggacccaagcatttgaatatgaagttgacccgtgctcaatttgaagggattgtcactgatctaatcagaaggactatcgctccatgccaaaaagctatgcaagatgcagaagtcagcaagagtgacataggagaagtgattcttgtgggtggcatgactaggatgcccaaggttcagcagactgtacaggatctttttggcagagccccaagtaaagctgtcaatcctgatgaggctgtggccattggagctgccattcagggaggtgtgttggccggcgatgtcacggatgtgctgctccttgatgtcactcccctgtctctgggtattgaaactctaggaggtgtctttaccaaacttattaataggaataccactattccaaccaagaagagccaggtattctctactgccgctgatggtcaaacgcaagtggaaattaaagtgtgtcagggtgaaagagagatggctggagacaacaaactccttggacagtttactttgattggaattccaccagcccctcgtggagttcctcagattgaagttacatttgacattgatgccaatgggatagtacatgtttctgctaaagataaaggcacaggacgtgagcagcagattgtaatccagtcttctggtggattaagcaaagatgatattgaaaatatggttaaaaatgcagagaaatatgctgaagaagaccggcgaaagaaggaacgagttgaagcagttaatatggctgaaggaatcattcacgacacagaaaccaagatggaagaattcaaggaccaattacctgctgatgagtgcaacaagctgaaagaagagatttccaaaatgagggagctcctggctagaaaagacagcgaaacaggagaaaatattagacaggcagcatcctctcttcagcaggcatcactgaagctgttcgaaatggcatacaaaaagatggcatctgagcgagaaggctctggaagttctggcactggggaacaaaaggaagatcaaaaggaggaaaaacagtaa

SEQ ID NO: 8 - protein, 679 aa, Homo sapiens
MISASRAAAARLVGAAASRGPTAARHQDSWNGLSHEAFRLVSRRDYASEAIKGAVVGIDLGTTNSCVAVMEGKQAKVLENAEGARTTPSVVAFTADGERLVGMPAKRQAVTNPNNTFYATKRLIGRRYDDPEVQKDIKNVPFKIVRASNGDAWVEAHGKLYSPSQIGAFVLMKMKETAENYLGHTAKNAVITVPAYFNDSQRQATKDAGQISGLNVLRVINEPTAAALAYGLDKSEDKVIAVYDLGGGTFDISILEIQKGVFEVKSTNGDTFLGGEDFDQALLRHIVKEFKRETGVDLTKDNMALQRVREAAEKAKCELSSSVQTDINLPYLTMDSSGPKHLNMKLTRAQFEGIVTDLIRRTIAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKMEEFKDQLPADECNKLKEEISKMRELLARKDSETGENIRQAASSLQQASLKLFEMAYKKMASEREGSGSSGTGEQKEDQKEEKQ

SEQ ID NO: 9 - DNA, 1968 bp, Mus musculus
atgatgaagttcactgtggtggcggcggcgttgctgctgctgggcgcggtgcgggccgaggaggaggacaagaaggaggatgtgggcacggtggtcggcatcgacttggggaccacctattcctgcgtcggtgtgttcaagaacggccgcgtggagatcatagccaacgatcagggcaaccgcatcacgccgtcgtatgtggccttcactcctgaaggggagcgtctgattggcgatgcggccaagaaccaactcacgtccaaccccgagaacacggtcttcgatgccaagcgcctcatcggacgcacttggaatgacccttcggtgcagcaggacatcaagttcttgccattcaaggtggttgaaaagaaaactaaaccgtacattcaagttgatattggaggtgggcaaaccaagacatttgccccagaagaaatttctgccatggttctcactaaaatgaaggagactgctgaggcgtatttgggaaagaaggttacccatgcagttgttactgtaccagcttacttcaatgatgcccagcgacaagcaaccaaagatgctggcactattgctggactgaatgtcatgaggatcatcaatgagcctacagcagctgctattgcatatggcctggataagagagagggagagaagaacatccttgtgtttgacctgggtggcggcaccttcgatgtgtctcttctcaccattgacaatggtgtctttgaagtggtggccactaatggagatactcacctgggtggggaagactttgatcagcgggtcatggaacacttcatcaagttgtacaaaaagaaaactggtaaagatgttaggaaagacaacagagctgtgcagaaactccggcgtgaggtagaaaaggctaagagagccttgtcttctcagcatcaagcaaggattgaaattgagtccttcttcgaaggagaagacttctcagagacccttactcgggccaaatttgaagagctgaacatggacctgttccgctctaccatgaagcctgtccagaaagtgttggaagactctgatctgaagaaatctgatattgatgaaattgttctggttggtggatctactcgaattccaaagattcagcaactggtgaaagagttcttcaatggcaaggagccatcccgtggcataaaccccgatgaggctgtagcctatggtgccgctgtccaggctggtgtcctctctggtgatcaggatacaggtgatctggtactgcttgatgtttgtccccttacacttggtattgaaactgtgggaggagtcatgacaaaactgattccaaggaacactgtggtacccaccaagaagtctcagatcttctccacggcttccgataatcagccaactgtaacaatcaaggtctatgaaggtgaacgacccctaacaaaagacaatcatcttctgggtacatttgatctgactggaattcctcctgctccccgtggagttccccagattgaagtcacttttgagatagatgttaatggtattctccgagtgacagctgaagacaaaggtacaggaaacaaaaacaaaatcacaattaccaatgaccaaaaccgcctgacacctgaagaaattgaaaggatggttaatgatgctgagaagtttgctgaggaagacaaaaagctcaaagagcgcattgacaccaggaatgaattggaaagctatgcttattctctcaagaaccagattggagataaagaaaagctgggaggtaaactttcttctgaagataaagaaaccatggaaaaagctgtagaggaaaagattgaatggctggaaagccaccaggatgcggacattgaagactttaaagccaaaaagaaggaactagaagaaattgttcagccaattatcagcaaactctatggaagtggaggccctcccccaactggtgaagaggatacatcagaaaaagatgagttgtag

SEQ ID NO: 10 - protein, 655 aa, Mus musculus
MMKFTVVAAALLLLGAVRAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFFEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSGGPPPTGEEDTSEKDEL

SEQ ID NO: 11 - DNA, 528 bp, Rattus norvegicus
atggacatagccatccaccacccctggatccggcgtcccttctttcctttccactccccaagccgcctctttgaccagttcttcggagagcacctgttggagtctgacctcttttctacagccacttccctgagccccttctaccttcggccaccctccttcctgcgggcacctagctggattgacactgggctctcagagatgcgtatggagaaggacaggttctctgtgaacctggacgtgaagcacttctctccagaggaactcaaagtcaaggttctgggagacgtgattgaggtgcacggcaagcacgaagagcgccaggacgaacatggcttcatctccagggagttccacaggaagtaccggatcccagccgacgtggatcctctcaccattacttcttccctgtcatcggatggagtcctcactgtgaatggaccaaggaaacaggcctctggccctgagcgcaccattcccatcacccgtgaagagaagcctgctgtcactgcagcccctaagaagtag

SEQ ID NO: 12 - protein, 175 aa, Rattus norvegicus
MDIAIHHPWIRRPFFPFHSPSRLFDQFFGEHLLESDLFSTATSLSPFYLRPPSFLRAPSWIDTGLSEMRMEKDRFSVNLDVKHFSPEELKVKVLGDVIEVHGKHEERQDEHGFISREFHRKYRIPADVDPLTITSSLSSDGVLTVNGPRKQASGPERTIPITREEKPAVTAAPKK

SEQ ID NO: 13 - DNA, 435 bp, Homo sapiens
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 14 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 15 - DNA, 435 bp, Homo sapiens
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagactatcacctttgaaagtttcaaagagaacctgaaggactttctgcttgtcatcccctttgactgctgggagccagtccaggagtga

SEQ ID NO: 16 - protein, 144 aa, Homo sapiens [sequence omitted]

SEQ ID NO: 17 - DNA, 426 bp, Mus musculus
atgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacattgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccagtccaaaaatga

SEQ ID NO: 18 - protein, 141 aa, Mus musculus
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 19 - DNA, 426 bp, Mus musculus
atgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacgttgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccaggccaaaaatga

SEQ ID NO: 20 - protein, 141 aa, Mus musculus
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 21 - DNA, 435 bp, Felis catus
atgtggctgcagaacctgcttttcctgaacactgtggtctgcagcatctctgcacccaccagttcacccagctctgtcactcggccctggcaacacgtggatgccatgaaggaggctctgagccttctgaacaacagtagtgaaataactgctgtgatgaatgaaacggtagaagtcgtctctgaaatgtttgaccctgaggagccgaaatgcctgcagactcacctaaagctgtacgagcagggcctacggggcagcctcatcagcctcaaggagcctctgagaatgatggccaaccattacaagcagcactgcccccttactccggaaacgccctgtgaaacccagactatcaccttcaaaaatttcaaagagaaactgaaggattttctgtttaacaacccctttgactgctggggaccagaccagaagtaa

SEQ ID NO: 22 - protein, 144 aa, Felis catus
MWLQNLLFLNTVVCSISAPTSSPSSVTRPWQHVDAMKEALSLLNNSSEITAVMNETVEVVSEMFDPEEPKCLQTHLKLYEQGLRGSLISLKEPLRMMANHYKQHCPLTPETPCETQTITFKNFKEKLKDFLFNNPFDCWGPDQK

SEQ ID NO: 23 - DNA, 384 bp, Rattus norvegicus
gcacccacccgctcacccaaccctgtcacccggccctggaagcatgtagatgccatcaaagaagctctgagcctcctaaatgacatgcgtgctctggagaacgaaaagaacgaagacgtagacatcatctctaatgagttctccatccagaggccgacatgtgtgcagacccgcctgaagctatacaagcagggtctacggggcaacctcaccaaactcaatggcgccttgaccatgatagccagccactaccagacgaactgccctccaaccccggaaactgactgtgaaatagaagtcaccacctttgaggatttcataaagaaccttaaaggctttctgtttgatatcccttttgactgctggaagccggtccagaaatga

SEQ ID NO: 24 - protein, 127 aa, Rattus norvegicus
APTRSPNPVTRPWKHVDAIKEALSLLNDMRALENEKNEDVDIISNEFSIQRPTCVQTRLKLYKQGLRGNLTKLNGALTMIASHYQTNCPPTPETDCEIEVTTFEDFIKNLKGFLFDIPFDCWKPVQK

SEQ ID NO: 25 - DNA, 435 bp, Homo sapiens; identical to SEQ ID NO: 15

SEQ ID NO: 26 - protein, 144 aa, Homo sapiens [sequence omitted]

SEQ ID NO: 27 - DNA, 1938 bp, Homo sapiens
agcttatggccaaagccgcggcgatcggcatcgacctgggcaccacctactcctgcgtgggggtgttccaacacggcaaggtggagatcatcgccaacgaccagggcaaccgcaccacccccagctacgtggccttcacggacaccgagcggctcatcggggatgcggccaagaaccaggtggcgctgaacccgcagaacaccgtgtttgacgcgaagcggctgatcggccgcaagttcggcgacccggtggtgcagtcggacatgaagcactggcctttccaggtgatcaacgacggagacaagcccaaggtgcaggtgagctacaagggggacaccaaggcattctaccccgaggagatctcgtccatggtgctgaccaagatgaaggagatcgccgaggcgtacctgggctacccggtgaccaacgcggtgatcaccgtgccggcctacttcaacgactcgcagcgccaggccaccaaggatgcgggtgtgatcgcggggctcaacgtgctgcggatcatcaacgagcccacggccgccgccatcgcctacggcctggacagaacgggcaagggggagcgcaacgtgctcatctttgacctgggcgggggcaccttcgacgtgtccatcctgacgatcgacgacggcatcttcgaggtgaaggccacggccggggacacccacctgggtggggaggactttgacaacaggctggtgaaccacttcgtggaggagttcaagagaaaacacaagaaggacatcagccagaacaagcgagccgtgaggcggctgcgcaccgcctgcgagagggccaagaggaccctgtcgtccagcacccaggccagcctggagatcgactccctgtttgagggcatcgacttctacacgtccatcaccagggcgaggttcgaggagctgtgctccgacctgttccgaagcaccctggagcccgtggagaaggctctgcgcgacgccaagctggacaaggcccagattcacgacctggtcctggtcgggggctccacccgcatccccaaggtgcagaagctgctgcaggacttcttcaacgggcgcgacctgaacaagagcatcaaccccgacgaggctgtggcctacggggcggcggtgcaggcggccatcctgatgggggacaagtccgagaacgtgcaggacctgctgctgctggacgtggctcccctgtcgctggggctggagacggccggaggcgtgatgactgccctgatcaagcgcaactccaccatccccaccaagcagacgcagatcttcaccacctactccgacaaccaacccggggtgctgatccaggtgtacgagggcgagagggccatgacgaaagacaacaatctgttggggcgcttcgagctgagcggcatccctccggcccccaggggcgtgccccagatcgaggtgaccttcgacatcgatgccaacggcatcctgaacgtcacggccacggacaagagcaccggcaaggccaacaagatcaccatcaccaacgacaagggccgcctgagcaaggaggagatcgagcgcatggtgcaggaggcggagaagtacaaagcggaggacgaggtgcagcgcgagagggtgtcagccaagaacgccctggagtcctacgccttcaacatgaagagcgccgtggaggatgaggggctcaagggcaagatcagcgaggccgacaagaagaaggtgctggacaagtgtcaagaggtcatctcgtggctggacgccaacaccttggccgagaaggacgagtttgagcacaagaggaaggagctggagcaggtgtgtaaccccatcatcagcggactgtaccagggtgccggtggtcccgggcctgggggcttcggggctcagggtcccaagggagggtctgggtcaggccccaccattgaggaggtagattagactagtg

SEQ ID NO: 28 - DNA, 434 bp, Homo sapiens
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagactatcacctttgaaagtttcaaagagaacctgaaggactttctgcttgtcatcccctttgactgctgggagccagtccaggagtg

SEQ ID NO: 29 - DNA, 649 bp, Homo sapiens
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacag

SEQ ID NO: 30 - DNA, 986 bp, Adenovirus
atgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataa

SEQ ID NO: 31 - DNA, 426 bp, Mus musculus; identical to SEQ ID NO: 19